(19) United States
(12) Patent Application Publication
(10) Pub. No.: US 2013/0022914 A1
(43) Pub. Date: Jan. 24, 2013

(54) HOMOADAMANTANE DERIVATIVE, METHOD FOR PRODUCING THE SAME AND PHOTOSENSITIVE MATERIALS FOR PHOTORESIST

(71) Applicants: Shinji Tanaka, Ichihara-shi (JP); Yoshitaka Uenoyama, Ichihara-shi (JP); Hidetoshi Ono, Ichihara-shi (JP); Naoya Kawano, Ichihara-shi (JP); Katsuki Ito, Ichihara (JP)

(72) Inventors: Shinji Tanaka, Ichihara-shi (JP); Yoshitaka Uenoyama, Ichihara-shi (JP); Hidetoshi Ono, Ichihara-shi (JP); Naoya Kawano, Ichihara-shi (JP); Katsuki Ito, Ichihara (JP)

(73) Assignee: IDEMITSU KOSAN CO., LTD., Tokyo (JP)

(21) Appl. No.: 13/638,979

(22) PCT Filed: Mar. 16, 2011
(86) PCT No.: PCT/JP2011/001532

§ 371 Date: Oct. 2, 2012

(30) Foreign Application Priority Data
Apr. 2, 2010 (JP) 2010-086352

(51) Int. Cl. C07D 313/06; G03F 7/004; G03F 7/20; C08F 122/20

(52) U.S. Cl. 430/270.1; 526/268; 430/325; 549/268

(57) ABSTRACT

A homoadamantane derivative represented by the following formula (I): wherein R¹ and R² are independently a hydrogen atom or a linear, branched or cyclic hydrocarbon group having 1 to 6 carbon atoms, x is a hydroxyl group or a halogen atom, and n and m are independently an integer of 0 to 3, provided that n and m are not simultaneously 0.

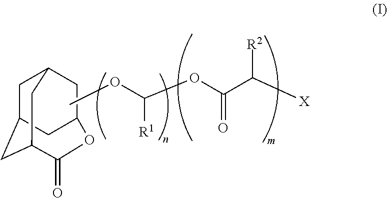

### TECHNICAL FIELD

The invention relates to a novel homoadamantane derivative, a (meth)acrylic ester, the production method thereof, a (meth)acrylic polymer, a positive photoresist composition, and a method for forming a photoresist pattern.

### BACKGROUND ART

In recent years, with progress in developing a semiconductor device which is smaller in size, a further decrease in size of a semiconductor device has been requested. Various methods for forming a fine pattern have been examined using photoresist materials corresponding to irradiation light with a short wavelength such as KrF, ArF or F₂ excimer-laser light, and a novel photoresist material which can correspond to irradiation light of short wavelength, such as excimer laser light has been desired.

As the photoresist material, many materials composed mainly of a phenol resin have conventionally been developed. Since these materials contain an aromatic ring, a large amount of light is absorbed, and hence, a pattern accuracy which is sufficient enough to correspond to a decrease in size cannot be obtained.

Under such circumstances, as the photoresist, a polymer obtained by copolymerizing a monomer compound having an alicyclic structure such as 2-methyl-2-adamanthylmethacrylate has been proposed as the photoresist used in the production of a semiconductor by means of an ArF excimer laser (Patent Document 1, for example).

With a further development in micromanufacturing technology, at this time, an attempt has been made to realize a stroke width of 32 nm or less. By the conventional technology, it has not been possible to satisfy the required performance such as adhesion with a substrate, exposure sensitivity, resolution, shape of pattern, depth of exposure and surface roughness. Specifically, problems in smoothness such as roughness of the pattern surface, i.e. LER and LWR, and a swell have come up to the surface. Further, by the recent method using liquid immersion exposure, insufficient development such as defects in a photoresist pattern caused by a liquid immersion medium is often seen. Further, in a semiconductor production process using extreme ultraviolet rays (EUV) with a wavelength of 13.5 nm, in order to improve throughput, development of a photoresist having a higher sensitivity has been desired.

Conventionally, in a photoresist for producing a semiconductor by means of an ArF excimer laser, a polymer obtained by copolymerizing various monomer compounds having cyclic lactone has been used in order to improve adhesion with a substrate. Under such circumstances, as lactone having a homoadamantane structure, 1-(5-oxo-4-oxa-5-homoadamantantyl)methacrylate has been proposed. A photosensitive composition having a high transparency, for short-wavelength light, high dry etching performance, capable of being developed in an alkaline solution, and which can form a photoresist pattern which has excellent adhesiveness and resolution, and a patterning method has been proposed (Patent Document 2, for example). However, including this homoadamantyl methacrylate compound, conventional monomer compounds having cyclic lactone do not have acid decomposition properties. Therefore, it does not function as a positive photoresist alone. Therefore, it is necessary to copolymerize with an acid-decomposable monomer such as tert-butyl methacrylate and 2-methyl-2-adamantyl methacrylate.

On the other hand, a photo acid generator (PAG) is an essential component in order to allow a positive photoresist to be subjected to photosensitive action (acid decomposition). Studies have been made to impart this PAG with acid-decomposition properties in order to improve surface roughness of a pattern surface called LER and LWR, which have come up to the surface with a decrease in size of a photoresist in recent years (Patent Document Nos. 3 to 6, for example). However, for a further improvement of the roughness, it is required to enhance the compatibility of a PAG in a photoresist resin and to disperse a PAG in a photoresist resin more homogeneously.

Further, in recent years, in the development of a low-molecular (single molecule) positive photoresist with an attempt to decreasing the roughness, an acid decomposition unit having various adamantane structure or various cyclic lactone structure has been actively introduced (Patent Documents 7 to 10, for example). However, with these technologies, satisfactory results have not yet been obtained.

### RELATED ART DOCUMENTS

Patent Document 1: JP-A-H4-39665

Patent Document 2: JP-A-2000-122294

Patent Document 3: JP-A-2009-149588

Patent Document 4: JP-A-2009-282494

Patent Document 5: JP-A-2008-69146

Patent Document 6: JP-T-2009-515944

Patent Document 7: JP-T-2009-527019

Patent Document 8: JP-A-2009-98448

Patent Document 9: JP-A-2009-223024

Patent Document 10: JP-A-2006-201762

The invention is aimed at providing a polymer which is excellent in roughness reduction, solubility, compatibility, defect reduction, exposure sensitivity or the like, when used as a positive photoresist, a monomer which generates such a polymer and a precursor thereof (intermediate, modifier).

According to the invention, the following homoadamantane derivative or the like are provided.
1. A homoadamantane derivative represented by the following formula (I):

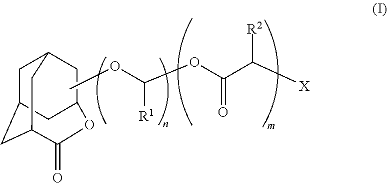

wherein R¹ and R² are independently a hydrogen atom or a linear, branched or cyclic hydrocarbon group having 1 to 6 carbon atoms, x is a hydroxyl group or a halogen atom, and n and m are independently an integer of 0 to 3, provided that n and m are not simultaneously 0; when n is 2 or more, plural R¹s may be the same or different, and when m is 2 or more, plural R²s may be the same or different.
2. The homoadamantane derivative according to 1, which is represented by any of the following formulas (1) to (3):

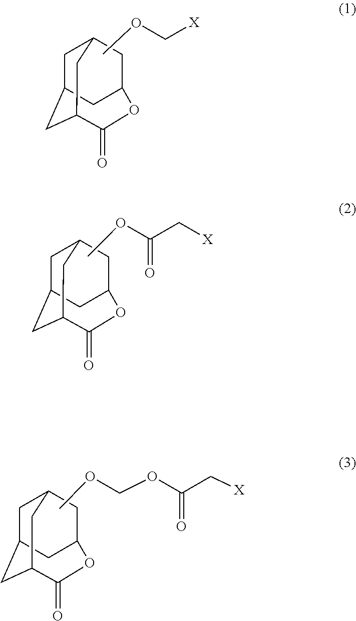

wherein X is a hydroxyl group or a halogen atom.
3. The homoadamantane derivative according to 2, which is represented by any of the following formulas (1a) to (3b):

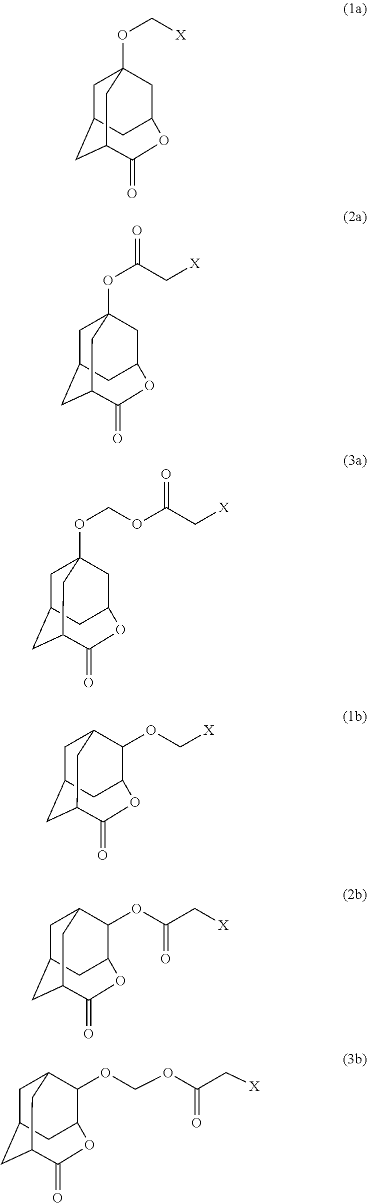

wherein X is a hydroxyl group or a halogen atom.
4. A method for producing a homoadmantane derivative according to any of 1 to 3 which comprises any of the following steps a to g:
a. reacting homoadamantyl alcohol represented by the following formula, aldehyde and a halogenated hydrogen gas;
b. reacting homoadamantyl alcohol represented by the following formula with alkylsulfoxide and an acid anhydride to obtain an alkylthioalkly ether, and reacting this alkylthioalkly ether with a halogenating agent;
c. reacting homoadmantyl alcohol represented by the following formula with 2-hydroxycarboxylic halide, 2-halogenated carboxylic halide or 2-halogenated carboxylic acid;
d. reacting the halogenated homoadamantane derivative obtained in any of above steps a to c with 2-hydroxycarboxylic acid; and
e. reacting the halogenated homoadamantane derivative obtained in any of above steps a to c with 2-halogenated carboxylic acid.

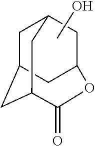

5. A (meth)acrylic ester represented by the following formula (II):

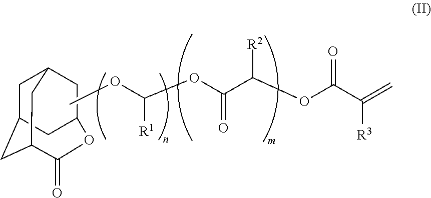

wherein R¹ and R² are independently a hydrogen atom or a linear, branched or cyclic hydrocarbon group having 1 to 6 carbon atoms and R³ represents a hydrogen atom, a methyl group or a trifluoromethyl group, n and m are independently an integer of 0 to 3, provided that n and m are not simultaneously 0 when n is 2 or more, plural R¹s may be the same or different, and when m is 2 or more, plural R²s may be the same or different.
6. The (meth)acrylic ester according to 5, which is represented by any of the following formulas (4) to (6):

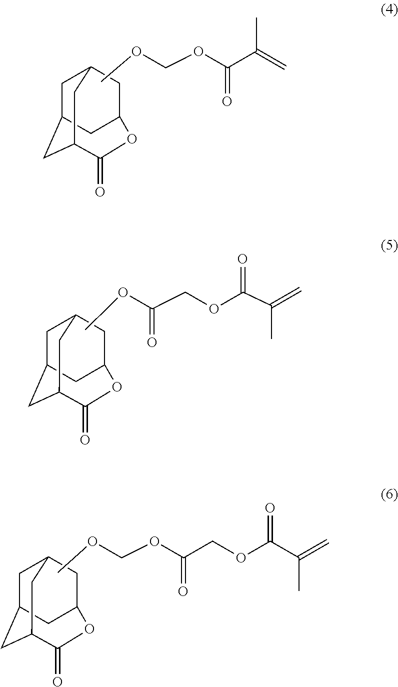

7. The (meth)acrylic ester according to 6, which is represented by any of the following formulas (4a) to (6b):

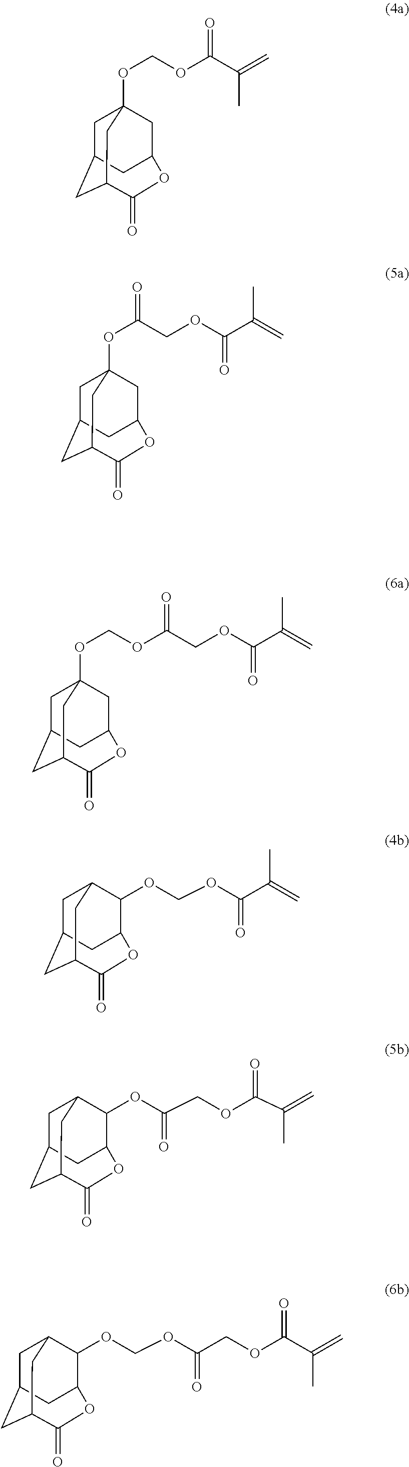 [SMILES: C=C(C)C(=O)OCOC12CC3CC(C1)OC(=O)C(C3)C2 ; C=C(C)C(=O)OCC(=O)OC12CC3CC(C1)OC(=O)C(C3)C2 ; C=C(C)C(=O)OCC(=O)OCOC12CC3CC(C1)OC(=O)C(C3)C2 ; C=C(C)C(=O)OCOC1C2CC3CC(C2)C(=O)OC1C3 ; C=C(C)C(=O)OCC(=O)OC1C2CC3CC(C2)C(=O)OC1C3 ; C=C(C)C(=O)OCC(=O)OCOC1C2CC3CC(C2)C(=O)OC1C3]

8. A method for producing the (meth)acrylic acid according to any of 5 to 7, wherein the homoadamantane derivative according to any of 1 to 3 is reacted with one or more selected from (meth)acrylic acid derivatives, halides of (meth)acrylic derivatives, anhydrides of (meth)acrylic derivatives and 2-hydroxyalkyl derivatives of (meth)acrylic acid derivatives.
9. A (meth)acrylic polymer obtained by polymerizing the (meth)acrylic esters according to any of 5 to 7.
10. A positive photoresist composition comprising the (meth)acrylic polymer according to 9 and a photoacid generator.
11. A method for forming a photoresist pattern comprising the steps of:
   forming on a substrate a photoresist film using the positive photoresist composition according to 10,
   subjecting the photoresist film to selective exposure, and
   subjecting the selectively exposed photoresist film to an alkaline development treatment to form a photoresist pattern.

According to the invention, it is possible to provide a polymer which is excellent in roughness reduction, solubility, compatibility, defect reduction, exposure sensitivity or the like, when used as a positive photoresist, and a monomer which generates such a polymer and a precursor thereof (intermediate, modifier).

### BRIEF DESCRIPTION OF THE DRAWINGS

FIG. 1 is a graph showing the polymerization speed of each monomer in Evaluation Example 1.

### MODE FOR CARRYING OUT THE INVENTION

The homoadamantane derivative of the invention is represented by the following formula (I):

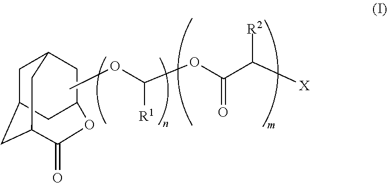

wherein R¹ and R² are independently a hydrogen atom or a linear, branched or cyclic hydrocarbon group having 1 to 6 carbon atoms; X is a hydroxyl group or a halogen atom, and n and m are each an integer of 0 to 3, provided that n and m are not simultaneously 0;

when n is 2 or more, plural R¹s may be the same or different, and when m is 2 or more, plural R²s may be the same or different.

R¹ and R² are preferably a hydrogen atom or a linear, branched or cyclic alkyl group having 1 to 6 carbon atoms. Examples of the alkyl group include a linear or branched alkyl group such as a methyl group, an ethyl group, an n-propyl group, an isopropyl group, an n-butyl group, a sec-butyl group, a tert-butyl group, an n-pentyl group, an isopentyl group and a hexyl group; and a cyclic structure such as a cyclopentyl ring and a cyclohexyl ring. As R¹ and R², a hydrogen atom and a methyl group are particularly preferable, with a hydrogen atom being particularly preferable.

As X, in addition to a hydroxyl group, a fluorine atom, a chlorine atom, a bromine atom and an iodine atom can be given. Of these, a hydroxyl group, a chlorine atom and a bromine atom are preferable.

As the combination of n and m in the formula (I), n and m are combined arbitrarily in an integer of 0 to 3. Of these, (n, m)=(0, 1), (0, 2), (1, 0), (1, 1), (1, 2), (2, 0), (2, 1), (2, 2) are preferable, with (n, m)=(0, 1), (0, 2), (1, 0) and (1, 1) being more preferable.

As for the position of a substituent on the homoadamantane structure in the formula (I), any position from 1 to 11 excluding 4 and 5 can be taken. However, in respect of easiness in synthesis, 1 or 2 is preferable.

It is preferred that the homoadamantane derivative of the invention be represented by any of the following formulas (1) to (3):

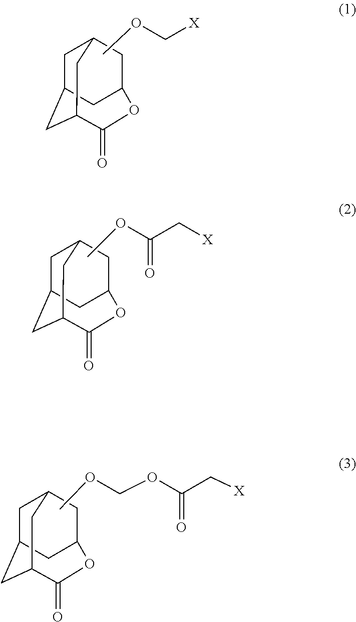

wherein X is a hydroxyl group or a halogen atom.

It is more preferred that the homoadamantane derivative of the invention be represented by any of the following formulas (1a) to (3b):

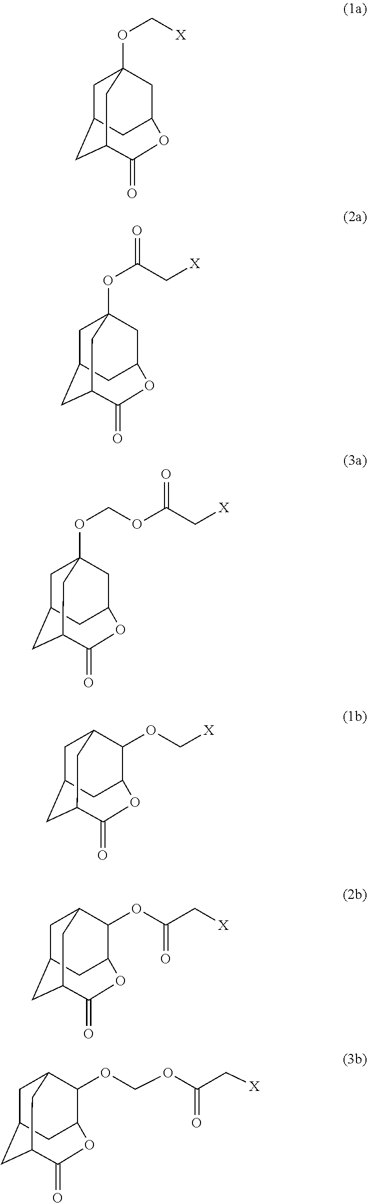

wherein X is a hydroxyl group or a halogen atom.

Specific examples of the homoadamantane derivative of the invention represented by the above-formula (I) include (5-oxo-4-oxa-5-homoadamantane-1-yl)oxymethanol, 1-(5-oxo-4-oxa-5-homoadamantane-1-yl)oxyethanol, 2-(5-oxo-4-oxa-5-homoadamantane-1-yl)oxy-2-oxoethanol, 2-(5-oxo-4-oxa-5-homoadamantane-1-yl)oxy-2-oxo-1-methylethanol, 2-(2-(5-oxo-4-oxa-5-homoadamantane-1-yl)oxy-2-oxoethoxy)-2-oxoethanol, 2-(2-(5-oxo-4-oxa-5-homoadamantane-1-yl)oxy-2-oxo-1-methylethoxy)-2-oxoethanol, 2-(2-(5-oxo-4-oxa-5-homoadamantane-1-yl)oxy-2-oxoethoxy)-2-oxo-1-methylethanol, 2-(2-(5-oxo-4-oxa-5-homoadamantane-1-yl)oxy-2-oxo-1-methylethoxy)-2-oxo-1-methylethanol, 2-(2-(5-oxo-4-oxa-5-homoadamantane-1-yl)oxy-2-oxo-1-ethylethoxy)-2-oxo-1-methylethanol, 2-(2-(5-oxo-4-oxa-5-homoadamantane-1-yl)oxy-2-oxo-1-methylethoxy)-2-oxo-1-ethylethanol, 2-(2-(5-oxo-4-oxa-5-homoadamantane-1-yl)oxy-2-oxo-1-ethylethoxy)-2-oxo-1-ethylethanol, 2-(5-oxo-4-oxa-5-homoadamantane-1-yl)oxymethoxy-2-oxoethanol, 2-(5-oxo-4-oxa-5-homoadamantane-1-yl)oxymethylmethoxy-2-oxoethanol, 2-(5-oxo-4-oxa-5-homoadamantane-1-yl)oxymethoxy-2-oxo-1-methylethanol, 2-(5-oxo-4-oxa-5-homoadamantane-1-yl)oxymethylmethoxy-2-oxo-1-methylethanol, 2-(5-oxo-4-oxa-5-homoadamantane-1-yl)oxyethylmethoxy-2-oxo-1-methylethanol, 2-(5-oxo-4-oxa-5-homoadamantane-1-yl)oxymethylmethoxy-2-oxo-1-ethylethanol, 2-(5-oxo-4-oxa-5-homoadamantane-1-yl)oxyethylmethoxy-2-oxo-1-ethylethanol, (5-oxo-4-oxa-5-homoadamantane-1-yl)oxymethylchloride, 1-(5-oxo-4-oxa-5-homoadamantane-1-yl)oxyethylchloride, 2-(5-oxo-4-oxa-5-homoadamantane-1-yl)oxy-2-oxoethylchloride, 2-(5-oxo-4-oxa-5-homoadamantane-1-yl)oxy-2-oxo-1-methylethylchloride, 2-(2-(5-oxo-4-oxa-5-homoadamantane-1-yl)oxy-2-oxo-ethoxy)-2-oxoethylchloride, 2-(2-(5-oxo-4-oxa-5-homoadamantane-1-yl)oxy-2-oxo-1-methylethoxy)-2-oxoethylchloride, 2-(2-(5-oxo-4-oxa-5-homoadamantane-1-yl)oxy-2-oxoethoxy)-2-oxo-1-methylethylchloride, 2-(2-(5-oxo-4-oxa-5-homoadamantane-1-yl)oxy-2-oxo-1-methylethoxy)-2-oxo-1-methylethylchloride, 2-(2-(5-oxo-4-oxa-5-homoadamantane-1-yl)oxy-2-oxo-1-ethylethoxy)-2-oxo-1-methylethylchloride, 2-(2-(5-oxo-4-oxa-5-homoadamantane-1-yl)oxy-2-oxo-1-methylethoxy)-2-oxo-1-ethylethylchloride, 2-(2-(5-oxo-4-oxa-5-homoadamantane-1-yl)oxy-2-oxo-1-ethylethoxy)-2-oxo-1-ethylethylchloride, 2-(5-oxo-4-oxa-5-homoadamantane-1-yl)oxymethoxy-2-oxoethylchloride, 2-(5-oxo-4-oxa-5-homoadamantane-1-yl)oxymethylmethoxy-2-oxoethylchloride, 2-(5-oxo-4-oxa-5-homoadamantane-1-yl)oxymethoxy-2-oxo-1-methylethylchloride, 2-(5-oxo-4-oxa-5-homoadamantane-1-yl)oxymethylmethoxy-2-oxo-1-methylethylchloride, 2-(5-oxo-4-oxa-5-homoadamantane-1-yl)oxyethylmethoxy-2-oxo-1-methylethylchloride, 2-(5-oxo-4-oxa-5-homoadamantane-1-yl)oxymethylmethoxy-2-oxo-1-ethylethylchloride, 2-(5-oxo-4-oxa-5-homoadamantane-1-yl)oxyethylmethoxy-2-oxo-1-ethylethylchloride, (5-oxo-4-oxa-5-homoadamantane-1-yl)oxymethylbromide, 1-(5-oxo-4-oxa-5-homoadamantane-1-yl)oxyethylbromide, 2-(5-oxo-4-oxa-5-homoadamantane-1-yl)oxy-2-oxoethylbromide, 2-(5-oxo-4-oxa-5-homoadamantane-1-yl)oxy-2-oxo-1-methylethylbromide, 2-(2-(5-oxo-4-oxa-5-homoadamantane-1-yl)oxy-2-oxoethoxy)-2-oxoethylbromide, 2-(2-(5-oxo-4-oxa-5-homoadamantane-1-yl)oxy-2-oxo-1-methylethoxy)-2-oxoethylbromide, 2-(2-(5-oxo-4-oxa-5-homoadamantane-1-yl)oxy-2-oxo-ethoxy)-2-oxo-1-methylethylbromide, 2-(2-(5-oxo-4-oxa-5-homoadamantane-1-yl)oxy-2-oxo-1-methylethoxy)-2-oxo-1-methylethylbromide, 2-(2-(5-oxo-4-oxa-5-homoadamantane-1-yl)oxy-2-oxo-1-ethylethoxy)-2-oxo-1-methylethylbromide, 2-(2-(5-oxo-4-oxa-5-homoadamantane-1-yl)oxy-2-oxo-1-methylethoxy)-2-oxo-1-ethylethylbromide, 2-(2-(5-oxo-4-oxa-5-homoadamantane-1-yl)oxy-2-oxo-1-ethylethoxy)-2-oxo-1-ethylethylbromide, 2-(5-oxo-4-oxa-5-homoadamantane-1-yl)oxymethoxy-2-oxoethylbromide, 2-(5-oxo-4-oxa-5-homoadamantane-1-yl)oxymethylmethoxy-2-oxoethylbromide, 2-(5-oxo-4-oxa-5-homoadamantane-1-yl)oxymethoxy-2-oxo-1-methylethylbromide, 2-(5-oxo-4-oxa-5-homoadamantane-1-yl)oxymethylmethoxy-2-oxo-1-methylethylbromide, 2-(5-oxo-4-oxa-5-homoadamantane-1-yl)oxyethylmethoxy-2-oxo-1-methylethylbromide, 2-(5-oxo-4-oxa-5-homoadamantane-1-yl)oxymethylmethoxy-2-oxo-1-ethylethylbromide, 2 0 2-(5-oxo-4-oxa-5-homoadamantane-1-yl)oxyethylmethoxy-2-oxo-1-ethylethylbromide, (5-oxo-4-oxa-5-homoadamantane-2-yl)oxymethanol, 1-(5-oxo-4-oxa-5-homoadamantane-2-yl)oxyethanol, 2-(5-oxo-4-oxa-5-homoadamantane-2-yl)oxy-2-oxoethanol, 2-(5-oxo-4-oxa-5-homoadamantane-2-yl)oxy-2-oxo-1-methylethanol, 2-(2-(5-oxo-4-oxa-5-homoadamantane-2-yl)oxy-2-oxoethoxy)-2-oxoethanol, 2-(2-(5-oxo-4-oxa-5-homoadamantane-2-yl)oxy-2-oxo-1-methylethoxy)-2-oxoethanol, 2-(2-(5-oxo-4-oxa-5-homoadamantane-2-yl)oxy-2-oxoethoxy)-2-oxo-1-methylethanol, 2-(2-(5-oxo-4-oxa-5-homoadamantane-2-yl)oxy-2-oxo-1-methylethoxy)-2-oxo-1-methylethanol, 2-(2-(5-oxo-4-oxa-5-homoadamantane-2-yl)oxy-2-oxo-1-ethylethoxy)-2-oxo-1-methylethanol, 2-(2-(5-oxo-4-oxa-5-homoadamantane-2-yl)oxy-2-oxo-1-methylethoxy)-2-oxo-1-ethylethanol, 2-(2-(5-oxo-4-oxa-5-homoadamantane-2-yl)oxy-2-oxo-1-ethylethoxy)-2-oxo-1-ethylethanol, 2-(5-oxo-4-oxa-5-homoadamantane-2-yl)oxymethoxy-2-oxo-ethanol, 2-(5-oxo-4-oxa-5-homoadamantane-2-yl)oxymethylmethoxy-2-oxoethanol, 2-(5-oxo-4-oxa-5-homoadamantane-2-yl)oxymethoxy-2-oxo-1-methylethanol, 2-(5-oxo-4-oxa-5-homoadamantane-2-yl)oxymethylmethoxy-2-oxo-1-methylethanol, 2-(5-oxo-4-oxa-5-homoadamantane-2-yl)oxyethylmethoxy-2-oxo-1-methylethanol, 2-(5-oxo-4-oxa-5-homoadamantane-2-yl)oxymethylmethoxy-2-oxo-1-ethylethanol, 2-(5-oxo-4-oxa-5-homoadamantane-2-yl)oxyethylmethoxy-2-oxo-1-ethylethanol, (5-oxo-4-oxa-5-homoadamantane-2-yl)oxymethylchloride, 1-(5-oxo-4-oxa-5-homoadamantane-2-yl)oxyethylchloride, 2-(5-oxo-4-oxa-5-homoadamantane-2-yl)oxy-2-oxoethylchloride, 2-(5-oxo-4-oxa-5-homoadamantane-2-yl)oxy-2-oxo-1-methylethylchloride, 2-(2-(5-oxo-4-oxa-5-homoadamantane-2-yl)oxy-2-oxoethoxy)-2-oxoethylchoride, 2-(2-(5-oxo-4-oxa-5-homoadamantane-2-yl)oxy-2-oxo-1-methylethoxy)-2-oxoethylchloride, 2-(2-(5-oxo-4-oxa-5-homoadamantane-2-yl)oxy-2-oxoethoxy)-2-oxo-1-methylethylchloride, 2-(2-(5-oxo-4-oxa-5-homoadamantane-2-yl)oxy-2-oxo-1-methylethoxy)-2-oxo-1-methylethylchloride, 2-(2-(5-oxo-4-oxa-5-homoadamantane-2-yl)oxy-2-oxo-1-ethylethoxy)-2-oxo-1-methylethylchloride, 2-(2-(5-oxo-4-oxa-5-homoadamantane-2-yl)oxy-2-oxo-1-methylethoxy)-2-oxo-1-ethylethylchloride, 2-(2-(5-oxo-4-oxa-5-homoadamantane-2-yl)oxy-2-oxo-1-ethylethoxy)-2-oxo-1-ethylethylchloride, 2-(5-oxo-4-oxa-5-homoadamantane-2-yl)oxymethoxy-2-oxoethylchloride, 2-(5-oxo-4-oxa-5-homoadamantane-2-yl)oxymethylmethoxy-2-oxoethylchloride, 2-(5-oxo-4-oxa-5-homoadamantane-2-yl)oxymethoxy-2-oxo-1-methylethylchloride, 2-(5-oxo-4-oxa-5-homoadamantane-2-yl)oxymethylmethoxy-2-oxo-1-methylethylchloride, 2-(5-oxo-4-oxa-5-homoadamantane-2-yl)oxyethylmethoxy-2-oxo-1-methylethylchloride, 2-(5-oxo-4-oxa-5-homoadamantane-2-yl)oxymethylmethoxy-2-oxo-1-ethylethylchloride, 2-(5-oxo-4-oxa-5-homoadamantane-2-yl)oxyethylmethoxy-2-oxo-1-ethylethylchloride, (5-oxo-4-oxa-5-homoadamantane-2-yl)oxymethylbromide, 1-(5-oxo-4-oxa-5-homoadamantane-2-yl)oxyethylbromide, 2-(5-oxo-4-oxa-5-homoadamantane-2-yl)oxy-2-oxo-ethylbromide, 2-(5-oxo-4-oxa-5-homoadamantane-2-yl)oxy-2-oxo-1-methylethylbromide, 2-(2-(5-oxo-4-oxa-5-homoadamantane-2-yl)oxy-2-oxoethoxy)-2-oxoethylbromide, 2-(2-(5-oxo-4-oxa-5-homoadamantane-2-yl)oxy-2-oxo-1-methylethoxy)-2-oxoethylbromide, 2-(2-(5-oxo-4-oxa-5-homoadamantane-2-yl)oxy-2-oxoethoxy)-2-oxo-1-methylethylbromide, 2-(2-(5-oxo-4-oxa-5-homoadamantane-2-y0oxy-2-oxo-1-methylethoxy)-2-oxo-1-methylethylbromide, 2-(2-(5-oxo-4-oxa-5-homoadamantane-2-yl)oxy-2-oxo-1-ethylethoxy)-2-oxo-1-methylethylbromide, 2-(2-(5-oxo-4-oxa-5-homoadamantane-2-yl)oxy-2-oxo-1-methylethoxy)-2-oxo-1-ethylethylbromide, 2-(2-(5-oxo-4-oxa-5-homoadamantane-2-yl)oxy-2-oxo-1-ethylethoxy)-2-oxo-1-ethylethylbromide, 2-(5-oxo-4-oxa-5-homoadamantane-2-yl)oxymethoxy-2-oxoethylbromide, 2-(5-oxo-4-oxa-5-homoadamantane-2-yl)oxymethylmethoxy-2-oxoethylbromide, 2-(5-oxo-4-oxa-5-homoadamantane-2-yl)oxymethoxy-2-oxo-1-methylethylbromide, 2-(5-oxo-4-oxa-5-homoadamantane-2-yl)oxymethylmethoxy-2-oxo-1-methylethylbromide, 2-(5-oxo-4-oxa-5-homoadamantane-2-yl)oxyethylmethoxy-2-oxo-1-methylethylbromide, 2-(5-oxo-4-oxa-5-homoadamantane-2-yl)oxymethylmethoxy-2-oxo-1-ethylethylbromide, and 2-(5-oxo-4-oxa-5-homoadamantane-2-yl)oxyethylmethoxy-2-oxo-1-ethylethylbromide.

Of these homoadamantane derivatives, in respect of performance, easiness in production or the like, (5-oxo-4-oxa-5-homoadamantane-1-yl)oxymethyl chloride, 2-(5-oxo-4-oxa-5-homoadamantane-1-yl)oxy-2-oxoethyl chloride, 2-(2-(5-oxo-4-oxa-5-homoadamantane-1-yl)oxy-2-oxoethoxy)-2-oxoethanol, 2-(5-oxo-4-oxa-5-homoadamantane-1-yl)oxymethoxy-2-oxoethyl chloride, (5-oxo-4-oxa-5-homoadamantane-2-yl)oxymethyl chloride, 2-(5-oxo-4-oxa-5-homoadamantane-2-yl)oxy-2-oxoethyl chloride, 2-(2-(5-oxo 4-oxa-5-homoadamantane-2-yl)oxy-2-oxoethoxy)-2-oxoethanol, 2-(5-oxo-4-oxa-5-homoadamantane-2-yl)oxymethoxy-2-oxoethyl chloride or the like are preferable.

Specific examples of the homoadamantane derivatives of the invention will be shown below. The invention is, however, not limited to those mentioned below.

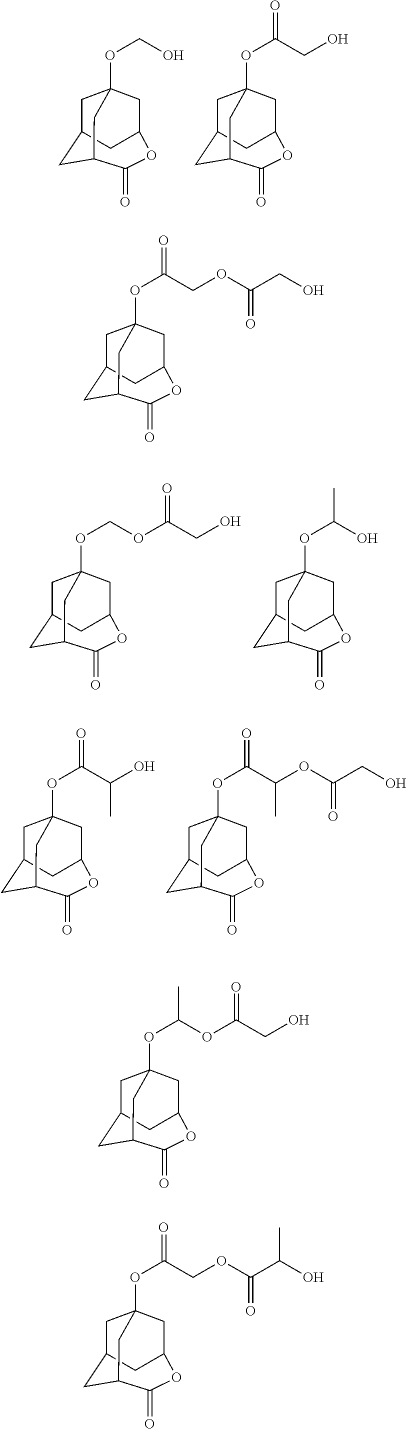

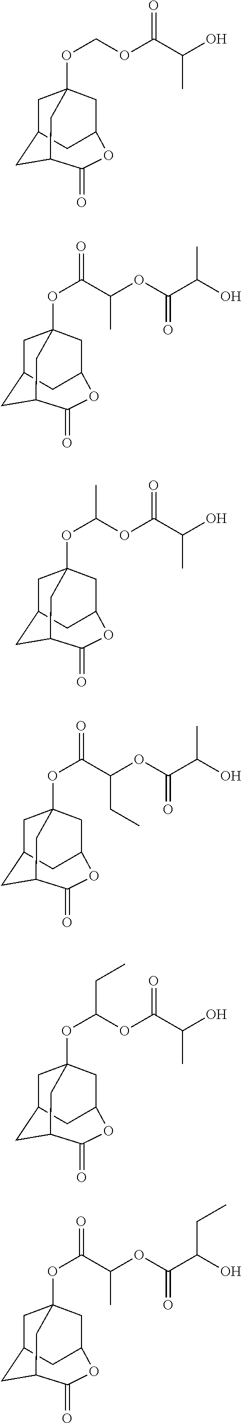

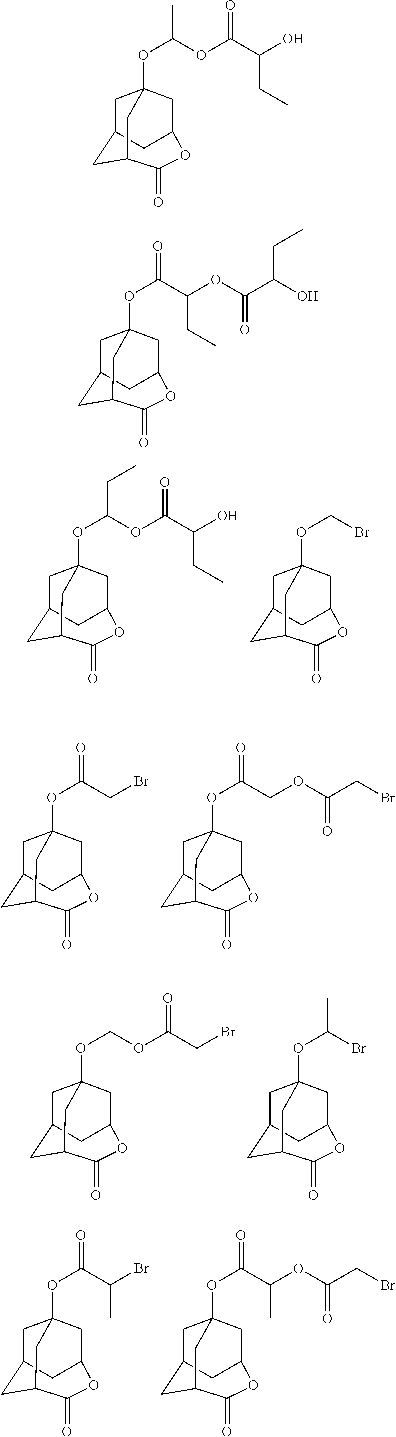

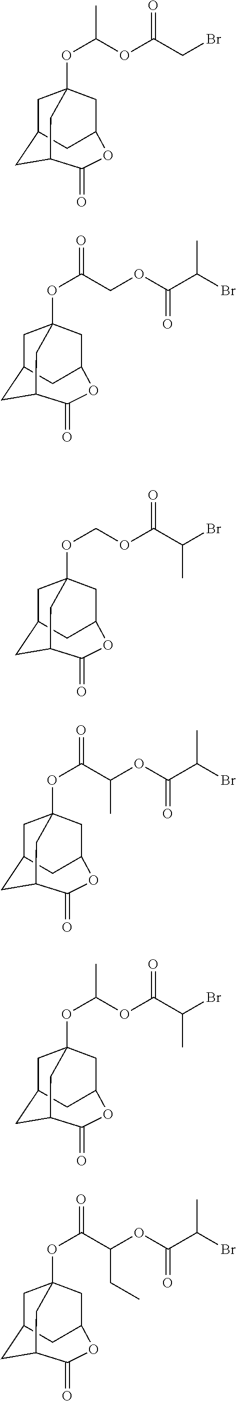

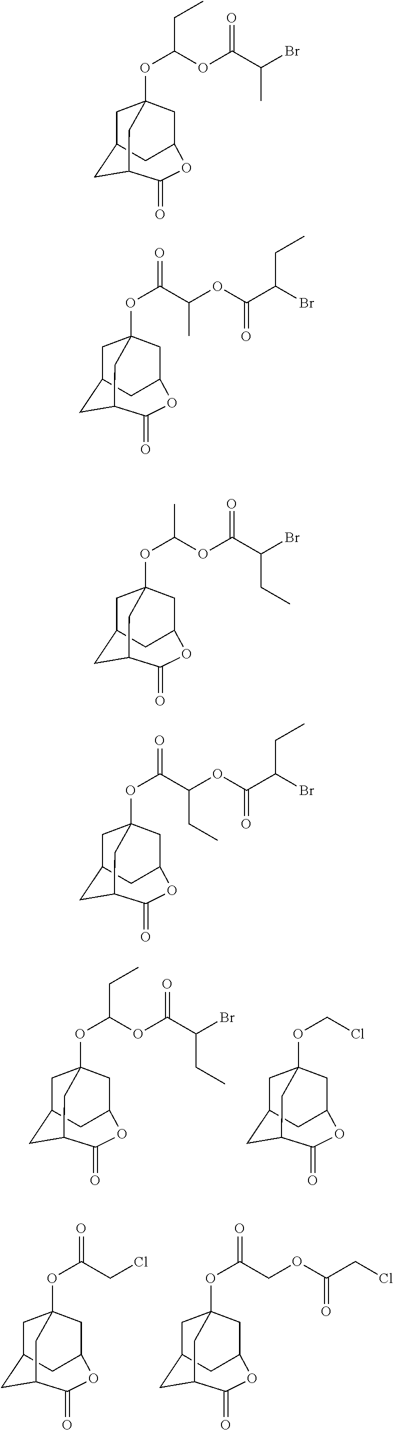

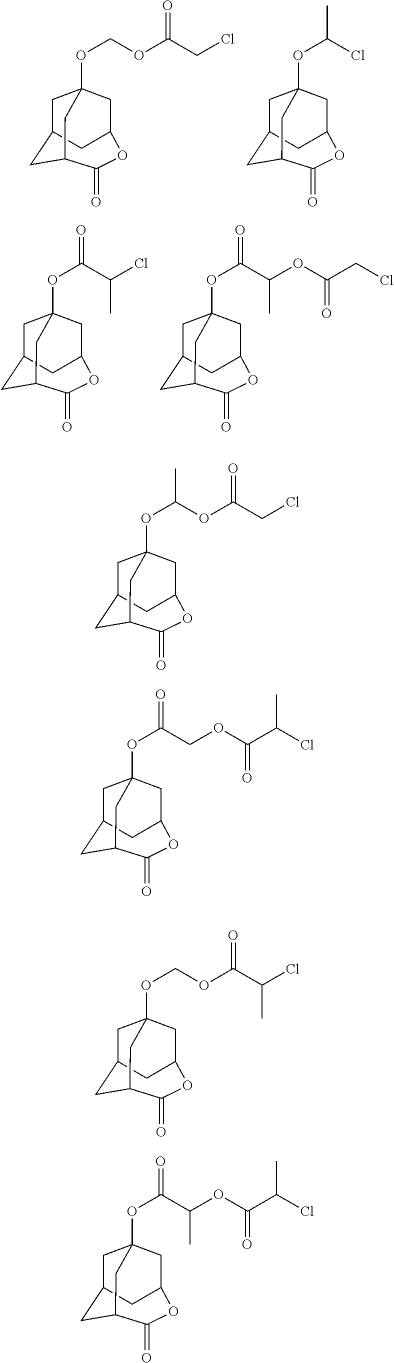

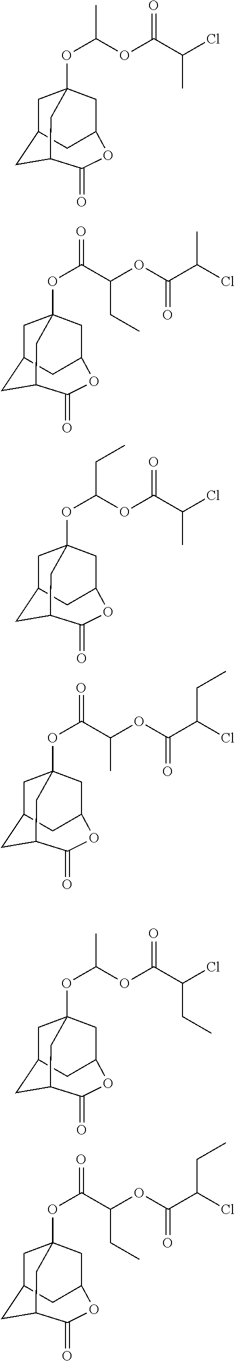

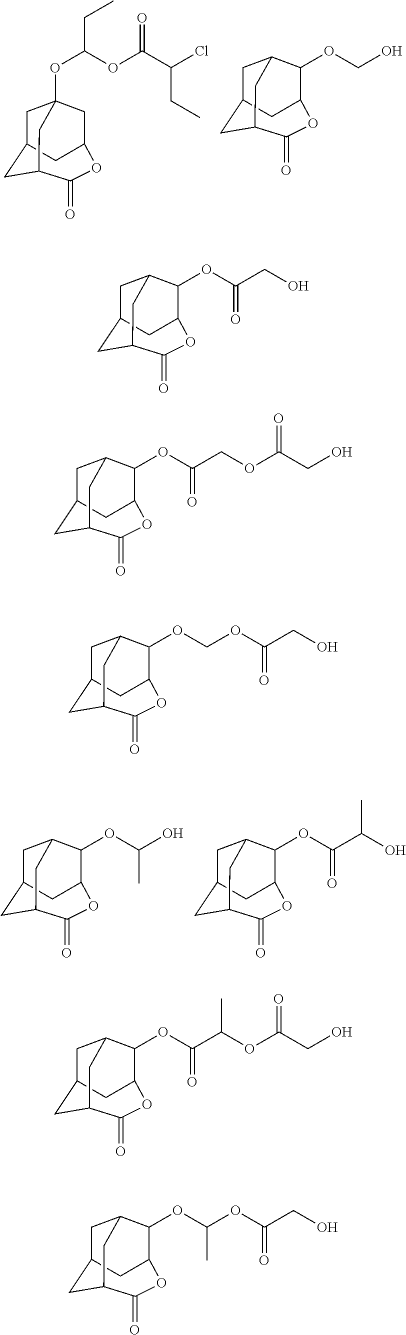

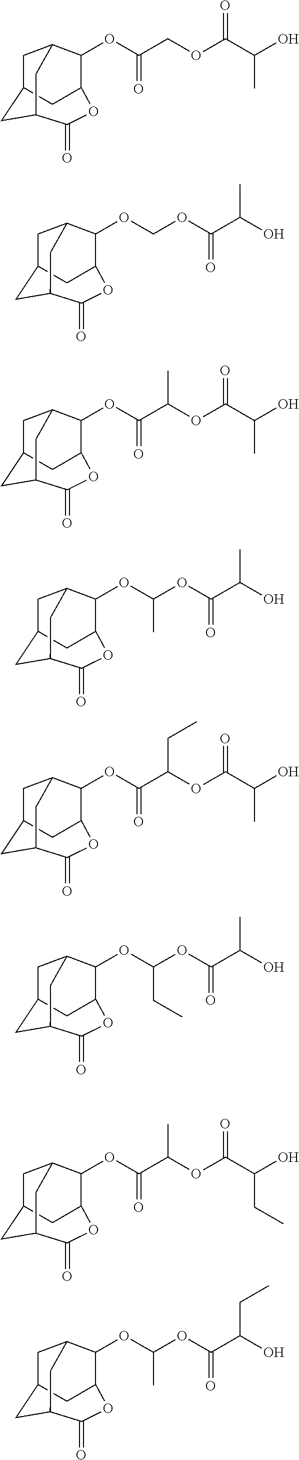

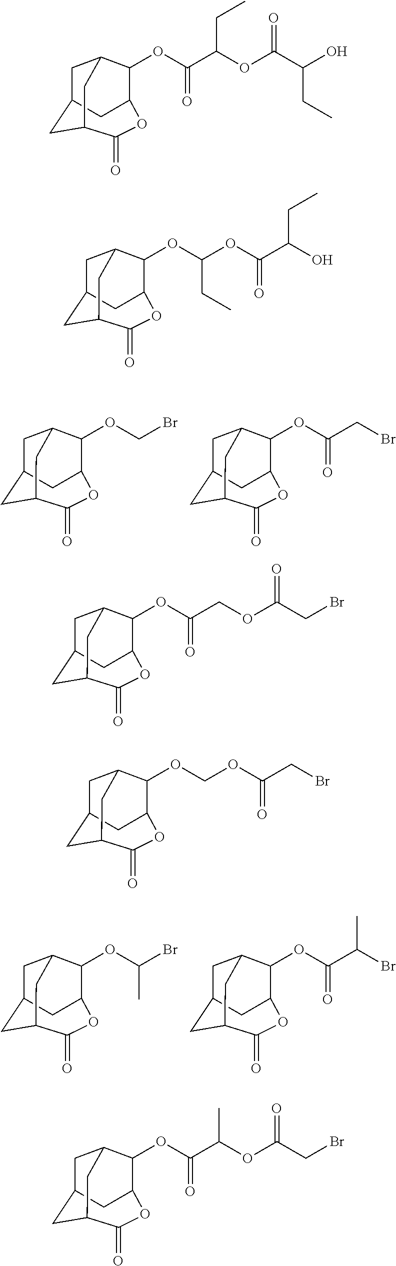

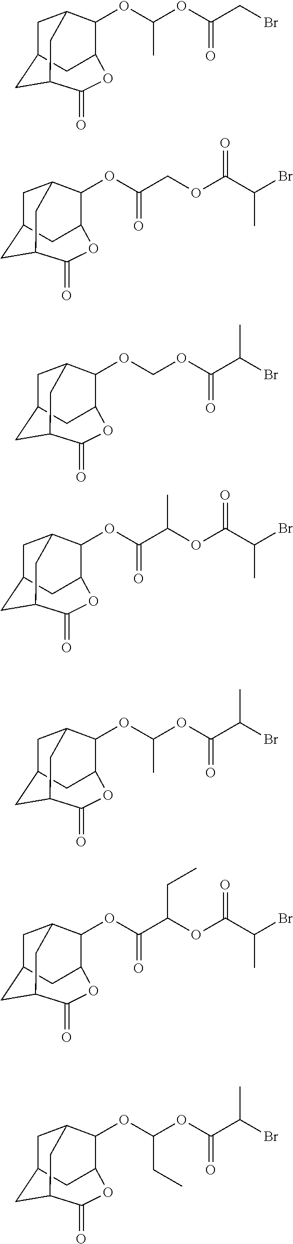

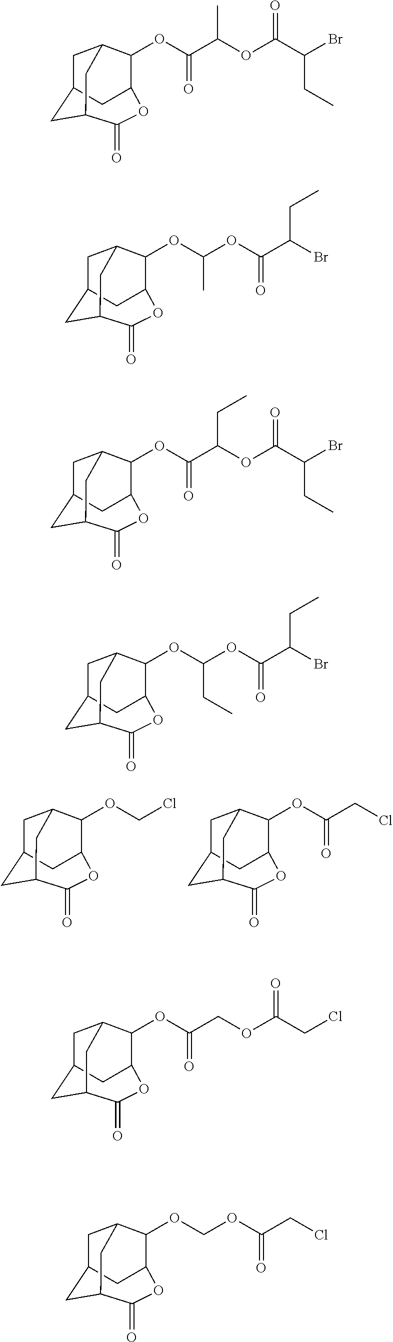

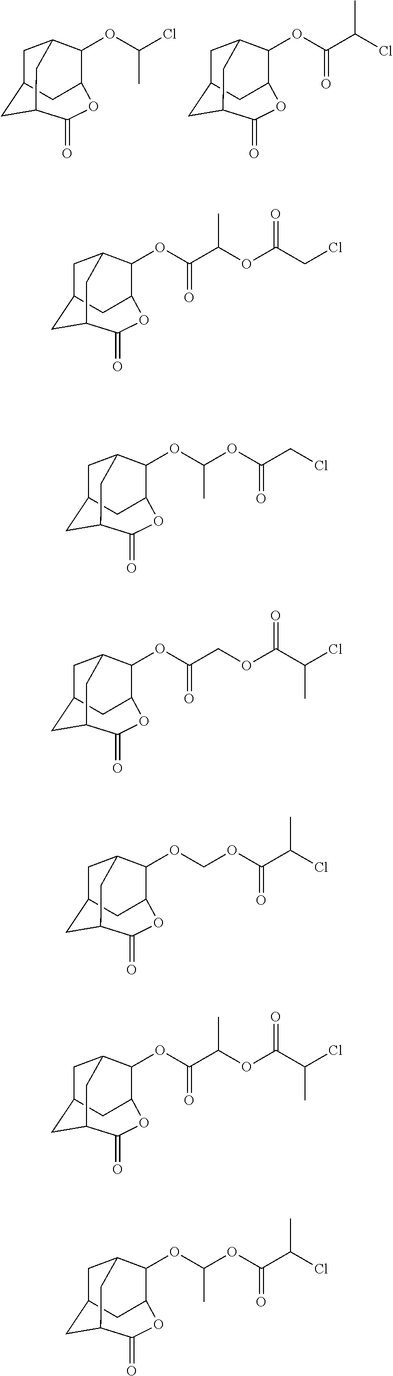

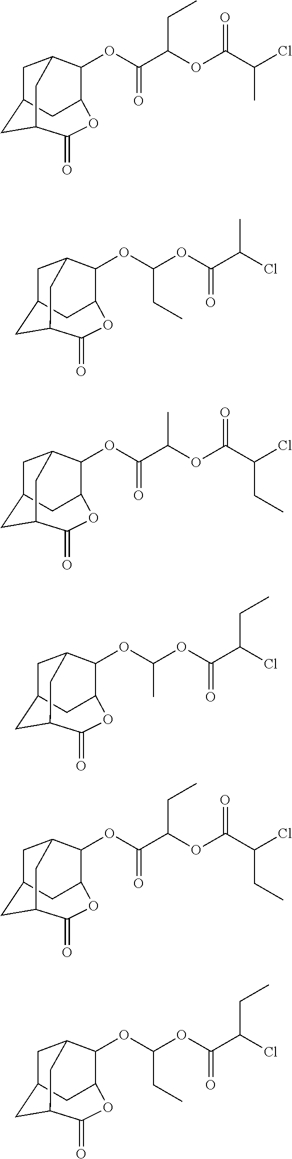 [SMILES: CCC(OC(=O)C(C)Cl)C(=O)OC1C2CC3CC(C2)C(=O)OC1C3 ; CCC(OC(=O)C(C)Cl)OC1C2CC3CC(C2)C(=O)OC1C3 ; CCC(Cl)C(=O)OC(C)C(=O)OC1C2CC3CC(C2)C(=O)OC1C3 ; CCC(Cl)C(=O)OC(C)OC1C2CC3CC(C2)C(=O)OC1C3 ; CCC(Cl)C(=O)OC(CC)C(=O)OC1C2CC3CC(C2)C(=O)OC1C3 ; CCC(OC(=O)C(Cl)CC)OC1C2CC3CC(C2)C(=O)OC1C3]

The homoadamantane derivatives of the invention can be produced by various methods. As representative examples, methods including the following steps will be given. The invention is, however, not limited to those mentioned above.

a. A step in which a homoadamantyl alcohol represented by the following formula is reacted by a halogenated hydrogen gas in the presence of aldehyde to obtain a homoadamantane derivative represented by the formula (I), which is a halogenated ether.

b. A step in which a homoadamantyl alcohol represented by the following formula is reacted in the presence of alkylsulfoxide and acid anhydride to obtain an alkylthioalkyl ether, and further this alkylthioalkyl ether is reacted with a halogenating agent to obtain a homoadamantane derivative represented by the formula (I), which is a halogenated ether.

c. A step in which homoadamantyl alcohol represented by the following formula is reacted with 2-hydrocarboxylic halide, 2-halogenated carboxylic halide or 2-halogenated carboxylic acid to obtain a homoadamantane derivative represented by the formula (I).

d. A step in which the halogenated homoadamantane derivative obtained in any of the above steps a to c is reacted with 2-hydroxycarboxylic acid

e. A step in which the halogenated homoadamantane derivative obtained in any of the above steps a to c is reacted with 2-halogenated carboxylic acid.

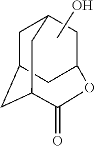

By repeating the same steps as the steps a and b, a compound having two or more “n”s can be obtained. By repeating the same steps as the steps c, d and e, a compound having two or more “m”s can be obtained.

As the aldehyde, a linear or branched aliphatic aldehyde such as formaldehyde, paraformaldehyde, acetaldehyde, propionaldehyde, butylaldehyde and isobutylaldehyde can be given, for example.

As the halogenated hydrogen gas, a hydrogen fluoride gas, a hydrogen chloride gas and a hydrogen bromide gas, or a mixture gas thereof can be given, for example.

As the alkylsulfoxide, symmetrical or asymmetrical alkyl sulfoxide such as dimethylsulfoxide, diethylsulfoxide, di-n-propylsulfoxide, diisopropylsulfoxide, di-n-butylsulfoxide, diisobutylsulofoxide, di-sec-butyl sulfoxide, di-tert-butyl sulfoxide, diisopentyl sulfoxide, methyl ethyl sulfoxide, methyl-tert-butyl sulfoxide can be given.

As the acid anhydride, an aliphatic or aromatic carboxylic anhydride, such as acetic anhydride, propionic anhydride, butyric anhydride, isobutyric anhydride, valeric anhydride, pivalic anhydride, benzoic anhydride, chloroacetic anhydride, and trifluoroacetic anhydride can be given, for example.

As the halogenating agent, a halogenated sulfur compound such as thionyl chloride, sulfuryl chloride, thionyl bromide, sulfuryl bromide, thionyl bromide chloride, sulfuryl bromide chloride and a halogenated phosphorous compound such as phosphorus trichloride, phosphorous tribromide, phosphorous triiodide, phosphorous trichloride, phosphorous tribromide, phosphorous pentachloride, and phosphorous pentabromide or the like can be given.

As the 2-hydroxycarboxylic acid, aliphatic-2-hydroxycarboxylic acid such as glycolic acid, lactic(2-hydroxypropionic acid), and 2-hydroxybutanoic acid and its acid anhydride can be given. As the 2-halogenated carboxylic acid, 2-halogenated aliphatic carboxylic acid such as 2-chloroacetic acid, 2-bromoacetic acid, 2-chloropionic acid and 2-bromopropionic acid and its acid anhydride can be given.

As the 2-hydroxylcarboxylic halide and the 2-halogenated carboxylic halide, a halide of 2-hydroxylcarboxylic acid and 2-halogenated carboxylic acid can be given.

The halogenated ether obtained in the step a can be obtained by reacting homoadamantyl alcohol with a halogenated hydrogen gas in the presence of an aldehyde. At this time, the reaction can be conducted in the presence or absence of an organic solvent.

No specific restrictions are imposed on the substrate concentration when an organic solvent is used as long as it is equal to or smaller than the saturated solubility of homoadamantyl alcohol. However, it is preferred that the substrate concentration be adjusted to be about 0.1 mol/L to 10 mol/L. A substrate concentration of 0.1 mol/L or more is economically advantageous since a necessary amount is obtained with a normal reactor. A substrate concentration of 10 mol/L or less is preferable since the temperature of the reaction liquid can be controlled easily.

As the usable organic solvent, a hydrocarbon-based solvent such as hexane, heptane, cyclohexane, ethyl cyclohexane, benzene, toluene and xylene, an ether-based solvent such as diethylether, dibutyl ether, THF (tetrahydrofuran), dioxane and DME (dimethoxy ethane), and a halogen-based solvent such as dichloromethane and carbon tetrachloride can be given. They can be used singly or in combination of two or more. A halogen-based solvent having a high dissolved amount of a halogenated hydrogen gas is preferable. Although the reaction temperature is arbitral, if the temperature is too high, the solubility of a halogenated hydrogen gas may be lowered. If the reaction temperature is too low, the reaction itself may proceed slowly. Therefore, the reaction temperature is preferably 0° C. to 40° C. The reaction pressure is arbitral. However, normal pressure is preferable since control of occurrence of a side reaction becomes necessary under pressurized conditions. If the pressure is too high, a special pressure-resistant apparatus becomes necessary, which results in economical disadvantage.

The alkylthioalkyl ether in the step b can be obtained by reacting homoadamantyl alcohol in the presence of alkylsulfoxide and an acid anhydride. At this time, the reaction can be conducted in the presence or absence of an organic solvent. However, normally, the reaction proceeds by using an excessive amount of alkylsulfoxide and an acid anhydride as a reaction reagent and as a solvent.

When another organic solvent is used separately, the organic solvent used and the pressure are the same as those in the step a. It is preferable to control so as to allow the substrate concentration to be about 1 mol/L to 10 mol/L. If the substrate concentration is 1 mol/L or more, it is economically advantageous since a necessary amount can be obtained in a normal reactor. If the substrate concentration is 10 mol/L or less, it is preferable since the temperature control of the reaction liquid becomes easy.

Although the reaction temperature is arbitral, if it is too high, lowering in selectivity may occur due to the occurrence of a side reaction, and it is too low, the speed of the reaction itself may become too slow. Therefore, the reaction temperature is preferably from room temperature to 60° C.

A halogenated alkyl ether can be obtained by reacting an alkylthioalkyl ether with a halogenating agent. This reaction may be conducted in the presence or absence of an organic solvent. However, a halogenating agent may be used in an excessive amount as the reaction reagent and as the solvent.

When an organic solvent is used separately, the substrate concentration, the organic solvent to be used and the pressure are the same as those in the step a.

Although the reaction temperature is arbitral, if it is too high, lowering in selectivity may occur due to the occurrence of a side reaction, and if it is too low, the speed of the reaction itself may become too slow. Therefore, the reaction temperature is preferably from room temperature to 100° C.

As for the esterification and the etherification in the steps a to c, a salt may be generated in a system by allowing a base to be acted on homoadmantyl alcohol and a reaction reagent. It is also possible to promote the reaction by forcibly removing water generated by azeotropic dehydration outside the system.

Although the above-mentioned esterification and etherification can be performed in the presence or absence of an organic solvent, when using an organic solvent, substrate concentration is the same as that in the above-mentioned process a.

As the organic solvent which can be used, in addition to the solvents exemplified in the above-mentioned process a, a non-protonic polar solvent such as DMF (N,N-dimethylformamide), DMSO (dimethylsulfoxide), NMP (N-methyl-2-pyrrolidone), HMPA (hexamethylphosphoric triamide), HMPT (hexamethylphosphorous triamide) and carbon bisulfide can be given, and they may be used singly or in a mixture of two or more.

As the above-mentioned base, in organic bases and organic amines such as sodium hydride, sodium hydroxide, potassium hydrate, sodium carbonate, potassium carbonate, sodium bicarbonate, potassium bicarbonate, silver oxide, sodium phosphate, potassium phosphate, disodium hydrogenphosphonate, dipotassium hydrogenphosphate, sodium dihydrogenphosphate, potassium dihydrogenphosphate, sodium methoxide, potassium tert-butoxide, triethylamine, tributylamine, trioctylamine, pyridine, N,N-dimethylamino pyridine, DBN (1,5-diazabicyclo [4,3,0]nona-5-en) and DBU (1,8-diazabicyclo [5,4,0]undeca-7-en) can be given.

In the case of azeotropic dehydration, as the solvent, a hydrocarbon system solvent such as cyclohexane, ethylcyclohexane, toluene and xylene are preferably selected. The amount ratio of a reaction reagent relative to homoadamantyl alcohol is about 0.01 to 100 times (mol), desirably 1 to 1.5 times (mol). The amount of a base to be added is about 0.1 to 10 times (mol), desirably about 1 to 1.5 times (mol) relative to homoadamantyl alcohol. The reaction temperature is about −200 to 200° C., preferably −50 to 100° C. The reaction pressure is about 0.01 to 10 MPa in terms of absolute pressure, preferably from normal pressure to 1 MPa. If the reaction time is long, the retaining time is prolonged. If the reaction pressure is too high, a specific pressure-resistant apparatus is required, resulting in an economical disadvantage.

In each of the above-mentioned reactions, after the reaction, the reaction liquid is separated into water and an organic phase. According to need, a generated product is extracted from an aqueous phase. By distilling off the solvent from the reaction liquid under reduced pressure, the homoadamantane derivative of the invention can be obtained. Purification may be conducted according to need. The reaction liquid may be subjected to a subsequent reaction without conducting purification. As for the purification method, a suitable method can be selected from common purification methods such as distillation, extraction washing, crystallization, activated carbon adsorption, and silica gel column chromatography taking into consideration the production scale and required purity. Extraction washing or crystallization is preferable since handling at a relatively low temperature is possible and a large amount of samples can be treated at once.

The (meth)acrylic ester of the invention is represented by the following formula (II):

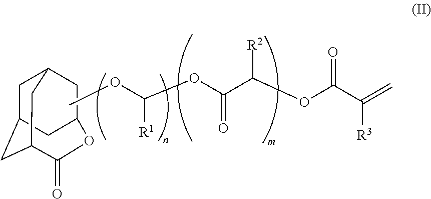

wherein R¹ and R² are independently a hydrogen atom or a straight-chain, branched or cyclic hydrocarbon group having 1 to 6 carbon atoms; R³ is a hydrogen atom, a methyl group or a trifluoromethyl group; n and m are independently an integer of 0 to 3, provided that n and m are not simultaneously 0.

When n is 2 or more, plural R¹s may be the same or different, and when m is 2 or more, plural R²s may be the same or different.

R³ in the formula (II) is preferably a hydrogen atom or a methyl group.

The meth(acrylic) ester of the invention is preferably represented by any of the following formulas (4) to (6):

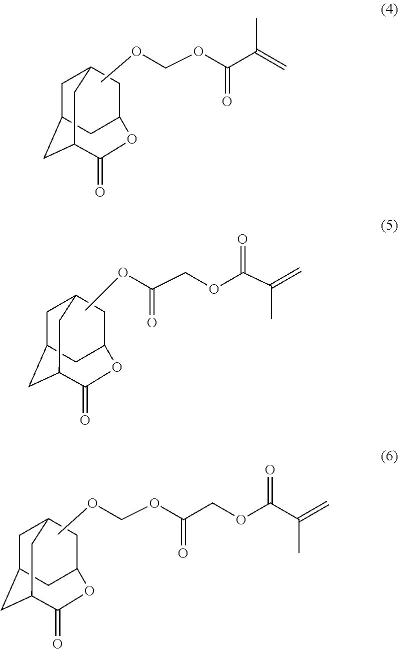

The meth(acrylic) ester of the invention is more preferably represented by any of the following formulas (4a) to (6b):

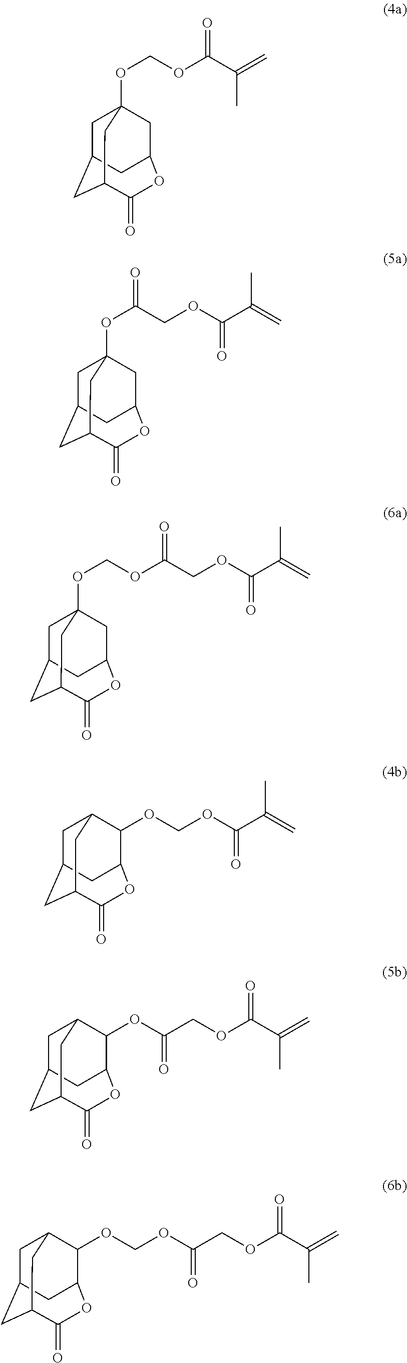

Specific examples of the (meth)acrylic acid ester of the invention represented by the formula (II) include 5-oxo-4-oxa-5-homoadamantane-1-yl)oxymethylmethacrylate, 1-(5-oxo-4-oxa-5-homoadamantane-1-yl)oxyethylmethacrylate, 2-(5-oxo-4-oxa-5-homoadamantane-1-yl)oxy-2-oxoethylmethacrylate, 2-(5-oxo-4-oxa-5-homoadamantane-1-yl)oxy-2-oxo-1-methylethylmethacrylate, 2-(2-(5-oxo-4-oxa-5-homoadamantane-1-yl)oxy-2-oxoethoxy)-2-oxoethylmethacrylate, 2-(2-(5-oxo-4-oxa-5-homoadamantane-1-yl)oxy-2-oxo-1-methylethoxy)-2-oxoethylmethacrylate, 2-(2-(5-oxo-4-oxa-5-homoadamantane1-yl)oxy-2-oxoethoxy)-2-oxo-1-methylethylmethacrylate, 2-(2-(5-oxo-4-oxa-5-homoadamantane-1-yl)oxy-2-oxo-1-methylethoxy)-2-oxo-1-methylethylmethacrylate, 2-(2-(5-oxo-4-oxa-5-homoadamantane-1-yl)oxy-2-oxo-1-ethylethoxy)-2-oxo-1-methylethylmethacrylate, 2-(2-(5-oxo-4-oxa-5-homoadamantane-1-yl)oxy-2-oxo-1-methylethoxy)-2-oxo-1-ethylethylmethacrylate, 2-(2-(5-oxo-4-oxa-5-homoadamantane1-yl)oxy-2-oxo-1-ethylethoxy)-2-oxo-1-ethylethylmethacrylate, 2-(5-oxo-4-oxa-5-homoadamantane-1-yl)oxymethoxy-2-oxoethylmethacrylate, 2-(5-oxo-4-oxa-5-homoadamantane-1-yl)oxymethylmethoxy-2-oxoethylmethacrylate, 2-(5-oxo-4-oxa-5-homoadamantane-1-yl)oxymethoxy-2-oxo-1-methylethylmethacrylate, 2-(5-oxo-4-oxa-5-homoadamantane-1-yl)oxymethylmethoxy-2-oxo-1-methylethylmethacrylate, 2-(5-oxo-4-oxa-5-homoadamantane-1-yl)oxyethylmethoxy-2-oxo-1-methylethylmethacrylate, 2-(5-oxo-4-oxa-5-homoadamantane-1-yl)oxymethylmethoxy-2-oxo-1-ethylethylmethacrylate, 2-(5-oxo-4-oxa-5-homoadamantane-1-yl)oxyethylmethoxy-2-oxo-1-ethylethylmethacrylate, (5-oxo-4-oxa-5-homoadamantane-2-yl)oxymethylmethacrylate, 1-(5-oxo-4-oxa-5-homoadamantane-2-yl)oxyethylmethacrylate, 2-(5-oxo-4-oxa-5-homoadamantane-2-yl)oxy-2-oxoethylmethacrylate, 2-(5-oxo-4-oxa-5-homoadamantane-2-yl)oxy-2-oxo-1-methylethylmethacrylate, 2-(2-(5-oxo-4-oxa-5-homoadamantane-2-yl)oxy-2-oxoethoxy)-2-oxoethylmethacrylate, 2-(2-(5-oxo-4-oxa-5-homoadamantane-2-yl)oxy-2-oxo-1-methylethoxy)-2-oxoethylmethacrylate, 2-(2-(5-oxo-4-oxa-5-homoadamantane-2-yl)oxy-2-oxoethoxy)-2-oxo-1-methylethylmethacrylate, 2-(2-(5-oxo-4-oxa-5-homoadamantane-2-yl)oxy-2-oxo-1-methylethoxy)-2-oxo-1-methylethylmethacrylate, 2-(2-(5-oxo-4-oxa-5-homoadamantane-2-yl)oxy-2-oxo-1-ethylethoxy)-2-oxo-1-methylethylmethacrylate, 2-(2-(5-oxo-4-oxa-5-homoadamantane-2-yl)oxy-2-oxo-1-methylethoxy)-2-oxo-1-ethylethylmethacrylate, 2-(2-(5-oxo-4-oxa-5-homoadamantane-2-yl)oxy-2-oxo-1-ethylethoxy)-2-oxo-1-ethylethylmethacrylate, 2-(5-oxo-4-oxa-5-homoadamantane-2-yl)oxymethoxy-2-oxoethylmethacrylate, 2-(5-oxo-4-oxa-5-homoadamantane-2-yl)oxymethylmethoxy-2-oxoethylmethacrylate, 2-(5-oxo-4-oxa-5-homoadamantane-2-yl)oxymethoxy-2-oxo-1-methylethylmethacrylate, 2-(5-oxo-4-oxa-5-homoadamantane-2-yl)oxymethylmethoxy-2-oxo-1-methylethylmethacrylate, 2-(5-oxo-4-oxa-5-homoadamantane-2-yl)oxyethylmethoxy-2-oxo-1-methylethylmethacrylate, 2-(5-oxo-4-oxa-5-homoadamantane-2-yl)oxymethylmethoxy-2-oxo-1-ethylethylmethacrylate, 2-(5-oxo-4-oxa-5-homoadamantane-2-yl)oxyethylmethoxy-2-oxo-1-ethylethylmethacrylate, (5-oxo-4-oxa-5-homoadamantane-1-yl)oxymethylacrylate, 2-(5-oxo-4-oxa-5-homoadamantane-1-yl)oxy-2-oxoethylacrylate, 2-(2-(5-oxo-4-oxa-5-homoadamantane-1-yl)oxy-2-oxoethoxy)-2-oxoethylacrylate, 2-(5-oxo-4-oxa-5-homoadamantane-1-yl)oxymethoxy-2-oxoethylacrylate, (5-oxo-4-oxa-5-homoadamantane-2-yl)oxymethylacrylate, 2-(5-oxo-4-oxa-5-homoadamantane-2-yl)oxy-2-oxoethylacrylate, 2-(2-(5-oxo-4-oxa-5-homoadamantane-2-yl)oxy-2-oxoethoxy)-2-oxoethylacrylate, 2-(5-oxo-4-oxa-5-homoadamantane-2-yl)oxymethoxy-2-oxoethylacrylate, (5-oxo-4-oxa-5-homoadamantane1-yl)oxymethyltrifluoromethylacrylate, 2-(5-oxo-4-oxa-5-homoadamantane-1-yl)oxy-2-oxoethyltrifluoromethylacrylate, 2-(2-(5-oxo-4-oxa-5-homoadamantane-1-yl)oxy-2-oxoethoxy)-2-oxoethyltrifluoromethylacrylate, 2-(5-oxo-4-oxa-5-homoadamantane-1-yl)oxymethoxy-2-oxoethyltrifluoromethylacrylate, (5-oxo-4-oxa-5-homoadamantane-2-yl)oxymethyltrifluromethylacrylate, 2-(5-oxo-4-oxa-5-homoadamantane-2-yl)oxy-2-oxoethyltrifluoromethylacrylate, 2-(2-(5-oxo-4-oxa-5-homoadamantane-2-yl)oxy-2-oxoethoxy)-2-oxoethyltrifluoromethylacrylate and 2-(5-oxo-4-oxa-5-homoadamantane-2-yl)oxymethoxy-2-oxoethyltrifluoromethylacrylate.

Of these (meth)acrylic acid esters, in respect of performance and easiness in production, 5-oxo-4-oxa-5-homoadamantane-1-yl)oxymethylmethacrylate, 2-(5-oxo-4-oxa-5-homoadamantane-1-yl)oxy-2-oxoethylmethacrylate, 2-(2-(5-oxo-4-oxa-5-homoadamantane-1-yl)oxy-2-oxoethoxy)-2-oxoethylmethacrylate, 2-(5-oxo-4-oxa-5-homoadamantane-1-yl)oxymethoxy-2-oxoethylmethacrylate, (5-oxo-4-oxa-5-homoadamantane-2-yl)oxymethylmethacrylate, 2-(5-oxo-4-oxa-5-homoadamantane-2-yl)oxy-2-oxoethylmethacrylate, 2-(2-(5-oxo-4-oxa-5-homoadamantane-2-yl)oxy-2-oxoethoxy)-2-oxoethylmethacrylate, 2-(5-oxo-4-oxa-5-homoadamantane-2-yl)oxymethoxy-2-oxoethylmethacrylate, or the like are preferable.

Specific examples of the (meth)acrylic esters of the invention are given below. The invention is not limited to those given below.

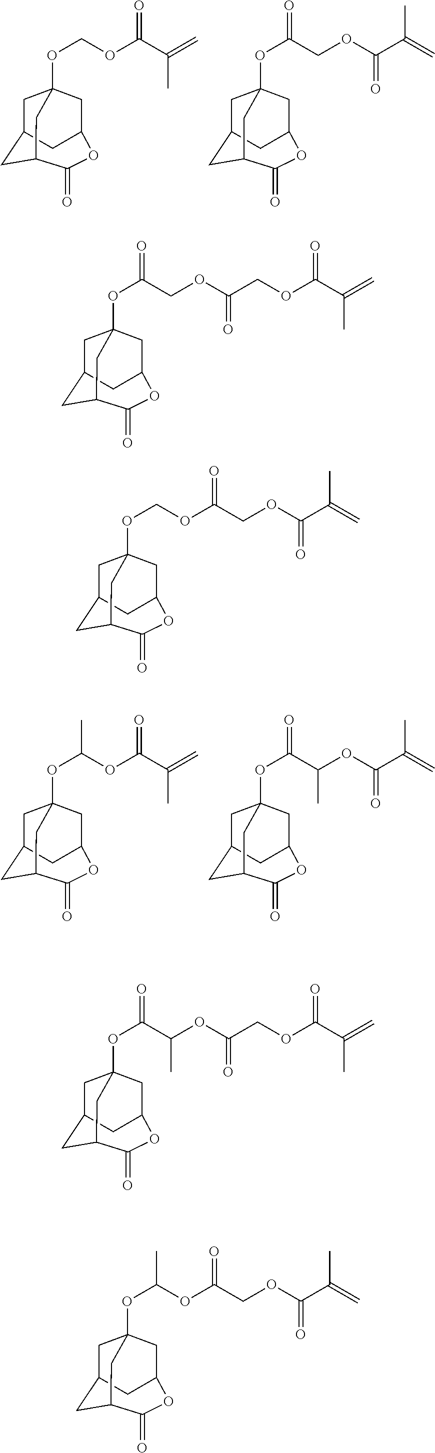

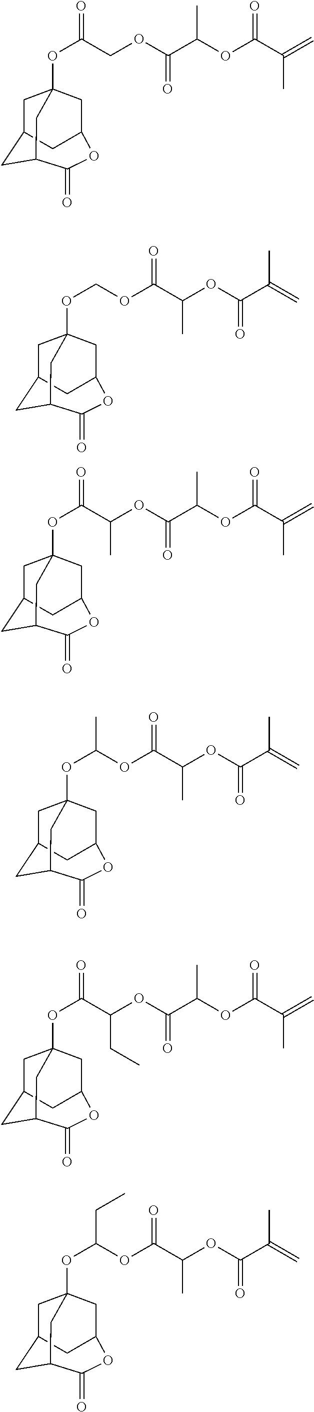

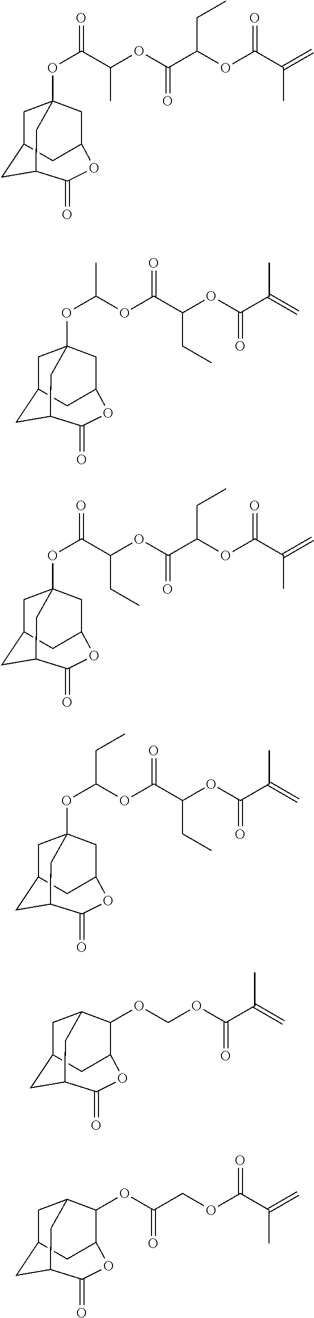

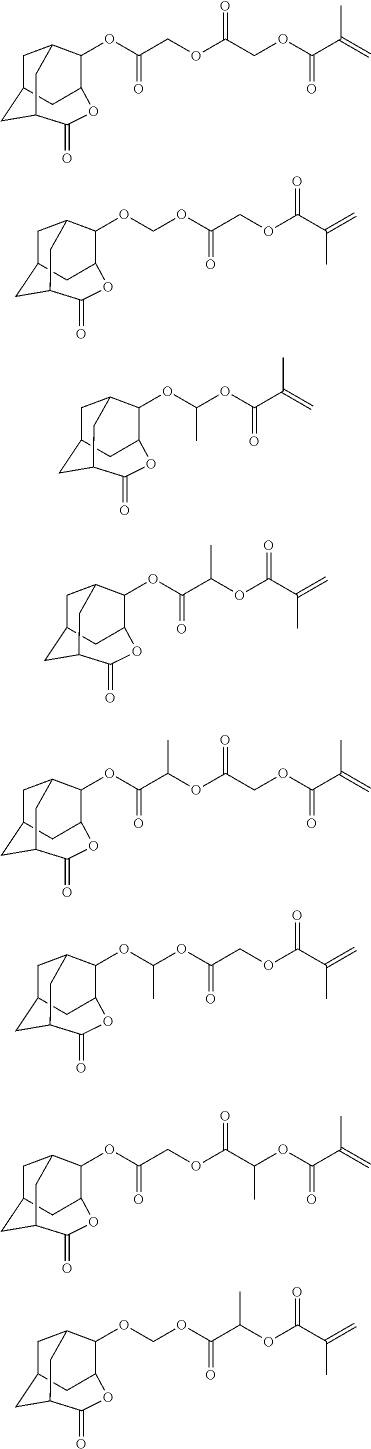

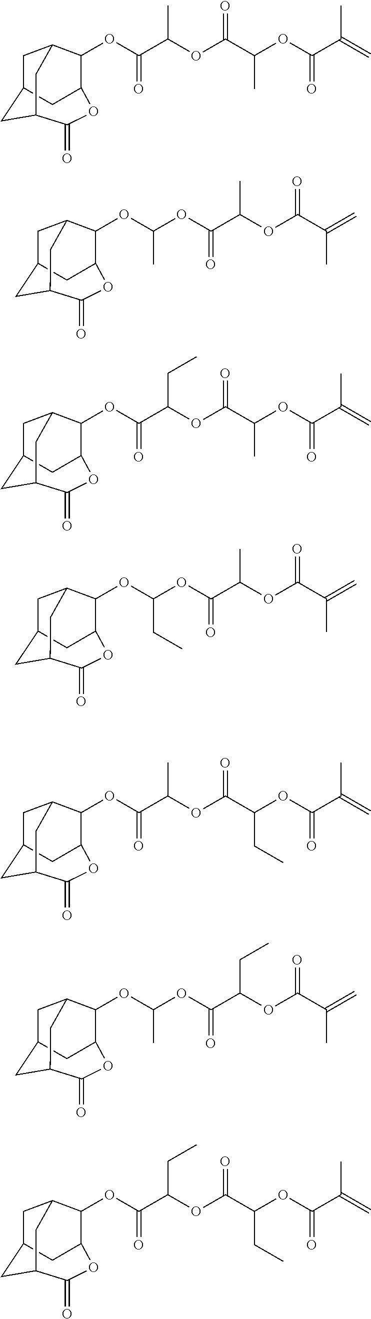

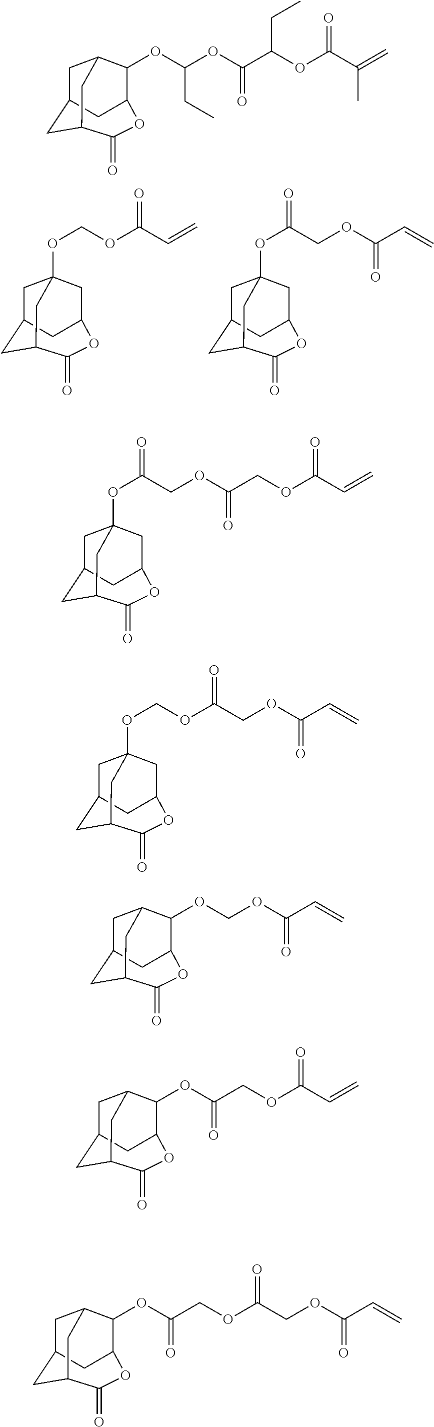

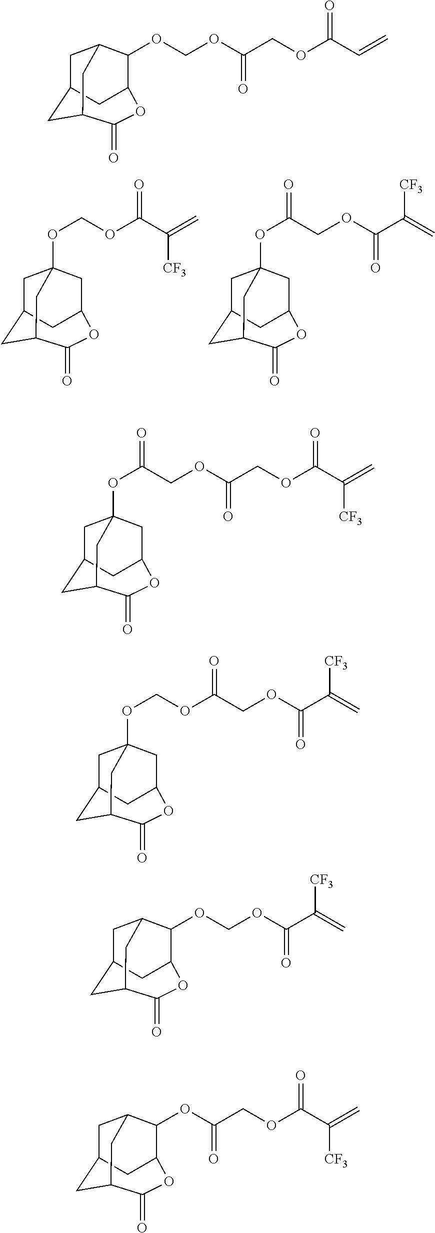

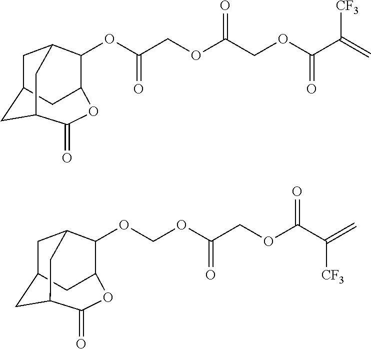 [SMILES: C=C(C(=O)OCC(=O)OCC(=O)OC1C2CC3CC(C2)C(=O)OC1C3)C(F)(F)F ; C=C(C(=O)OCC(=O)OCOC1C2CC3CC(C2)C(=O)OC1C3)C(F)(F)F]

The (meth)acrylic esters of the invention can be produced by various methods, and the methods are not particularly restricted. For example, the (meth)acrylic esters of the invention can be produced by the following methods.

A homoadamantane derivative represented by the formula (I) and one or more compounds selected from (meth)acrylic acid derivatives, halides of (meta)acrylic acid derivatives, anhydrides of (meth)acrylic acid derivatives and 2-hydroxyalkyl derivatives of (meth)acrylic acid derivatives (hereinafter, simply referred to as (meth)acrylic acid derivatives) are esterified to obtain (meth)acrylic esters represented by the formula (II).

As the (meth)acrylic acid derivatives, halogenated (meta)acrylic acid, such as acrylic acid, methacrylic acid, 2-fluoroacrylic acid and 2-trifluoromethylacrylic acid, or the like can be given, for example.

Examples of the halides of (meth)acrylic acid derivatives include acrylic fluoride, acrylic chloride, acrylic bromide, acrylic iodide, methacrylic fluoride, methacrylic chloride, methacrylic bromide, methacrylic iodide, 2-fluoroacrylic fluoride, 2-fluoroacylic chloride, 2-fluoroacrylic bromide, 2-fluoroacrylic iodide, 2-trifluoromethylacrylic fluoride, 2-trifluoromethylacrylic chloride, 2-trifluoromethylacrylic bromide, 2-trifluoromethylacrylic iodide or the like can be given.

As the anhydride of meth(acrylic) acid derivatives, acrylic anhydride, methacrylic anhydride, 2-fluoroacrylic anhydride, 2-trifluoromethylacrylic anhydride or the like can be given.

As the 2-hydroxyalkyl derivatives of (meth)acrylic acid derivatives, 2-hydroxyethyl acrylate, 2-hydroxyethyl methacrylate, 2-hydroxypropyl acrylate, 2-hydroxypropyl methacrylate or the like can be given.

Esterification is conducted by acting a base on the homoadamantane derivative represented by the formula (I) and the (meth)acrylic acid derivative to generate a salt in the system, or a reaction can be promoted by forcibly removing water generated by azeotropic dehydration outside the system.

Esterification can be conducted in the presence or absence of an organic solvent. When an organic solvent is used, it is preferred that the base concentration be about 0.1 mol/L to 10 mol/L. If the base concentration is 0.1 mol/L or more, it is economically preferable since a necessary amount can be obtained in a normal reaction apparatus. If a base concentration of 10 mol/L or less is preferable since the temperature control of the reaction liquid becomes easy.

As the usable organic solvent, a hydrocarbon-based solvent such as hexane, heptane, cyclohexane, ethylcyclohexane, benzene, toluene and xylene, an ether-based solvent such as diethyl ether, dibutyl ether, THF, dioxane and DME, a halogen-based solvent such as dichloromethane and carbon tetrachloride and a non-protonic polar solvent such as DMF, DMSO, NMP, HMPA, HMPT and carbon disulfide. These solvents may be used singly or in a mixture of two or more.

As the base, an inorganic base or an organic amine such as sodium hydride, sodium hydroxide, potassium hydroxide, sodium carbonate, potassium carbonate, sodium bicarbonate, potassium bicarbonate, silver oxide, sodium phosphate, potassium phosphate, disodium hydrogen phosphate, dipotassium hydrogen phosphate, sodium dihydrogen phosphate, potassium dihydrogen phosphate, sodium methoxide, potassium t-buthoxide, triethylamine, tributylamine, trioctylamine, pyridine, N,N-dimethylamino pyridine, DBN (1,5-diazabicyclo[4,3,0]nona-5-en), DBU (1,8-diazabicyclo[5,4,0]undeca-7-en) or the like are used.

In the case of an azeotropic dehydration reaction, the solvent is preferably a hydrocarbon-based solvent such as cyclohexane, ethylcyclohexane, toluene and xylene. The amount ratio of the reaction reagent relative to the alcohol having an alicyclic structure is about 0.01 to 100 times mol, desirably 1 to 1.5 times mol. The amount of a base to be added is about 0.1 to 10 times mol, desirably about 1 to 1.5 times mol relative to the alcohol having an alicyclic structure.

It suffices that the reaction temperature is about −200 to 200° C., preferably −50 to 100° C. The reaction pressure is about 0.01 to 10 MPa, preferably normal pressure to 1 MPa in terms of absolute pressure. If the reaction time is long, the retention time is long. If the pressure is too high, a special pressure-resistant apparatus becomes necessary, resulting in economical disadvantage.

After the reaction, the reaction liquid is separated into water and an organic phase. According to need, a reaction product is extracted from the aqueous phase. By distilling the solvent off from the reaction liquid under reduced pressure, the homoadamantane derivative of the invention can be obtained. The reaction liquid may be purified according to need, or may be used in the subsequent reaction without purification. As the purification method, a suitable method can be selected from general purification methods such as evaporation, extraction/washing, crystallization, adsorption by activated carbon and silica gel column chromatography taking into consideration the scale of production and required purity. Purification by extraction/washing or crystallization is preferable since handling at a relatively low temperature is possible and a large amount of samples can be treated at once.

The (meth)acrylic polymer of the invention can be obtained by polymerizing the (meth)acrylic ester represented by the formula (II).

The (meth)acrylic polymer of the invention may be a polymer having a repeating unit derived from one or more meth(acrylic)esters of the invention. It may be a homopolymer using only one (meth)acrylic ester, a copolymer using two or more (meth)acrylic esters, or a copolymer of one or more (meth)acrylic ester and other monomers.

As the (meth)acrylic polymer of the invention, one containing 10 to 90 mol %, more preferably 25 to 75 mol %, of the repeating unit derived from the (meth)acrylic acid ester represented by the formula (II).

No restrictions are imposed on the polymerization method, and polymerization can be conducted by a common polymerization method. For example, a polymerization method such as solution polymerization (polymerization at boiling point, polymerization below boiling point), emulsion polymerization, suspension polymerization, block polymerization or the like. A smaller amount of an un-reacted monomer having a high boiling point remaining in a reaction liquid after the polymerization is preferable. It is preferred that, at polymerization or after polymerization, it is preferred that an operation for removing an unreacted monomer be conducted according to need at the time of polymerization or after polymerization.

Of the above-mentioned polymerization methods, a polymerization reaction using a radical polymerization initiator in a solvent is preferable. Although no specific restrictions are imposed on the polymerization initiator, a peroxide-based polymerization initiator, an azo-based polymerization initiator or the like can be used.

As the peroxide-based polymerization initiator, organic peroxides such as peroxycarbonate, ketone peroxide, peroxide ketal, hydroperoxide, dialkyl peroxide, diacyl peroxide and peroxyester (lauroyl peroxide, benzoyl peroxide) can be given. As the azo-based polymerization initiator, azo compounds such as 2,2′-azobis isobutyronitrile, 2,2′-azobis (2-methylbutyronitrile), 2,2′-azobis(2,4-dimethylvarelonitrile), and dimethyl 2,2′-azobis(isobutyrate) can be given.

As for the above-mentioned polymerization initiator, one or two or more polymerization initiators may be used appropriately according to reaction conditions such as polymerization temperature.

After completion of the polymerization, the (meth)acrylic esters or other comonomers used can be removed from the produced polymer by various methods. In respect of operability or economy, a method in which an acrylic polymer is washed by using a poor solvent for an acrylic polymer is preferable. Of the poor solvents for an acrylic polymer, one having a low boiling point is preferable. Representative examples include methanol, ethanol, n-hexane and n-heptane.

As mentioned above, it is possible to obtain the (meth)acrylic ester represented by the formula (II) from the homoadamantane derivative represented by the formula (I), and further, it is possible to obtain a (meth)acrylic polymer by polymerizing the (meth)acrylic esters represented by the formula (II).

The (meth)acrylic polymers of the invention can be used as a positive photoresist. That is, from a highly reactive homoadamantane derivative represented by the formula (I), a homoadamantane structure can be introduced to a PAG, a low-molecular positive photoresist or a positive photoresist monomer. A homoadmantane structure can be further introduced to a positive photoresist polymer.

A carbon-carbon double bond contained in the (meth)acrylic esters represented by the formula (II) can promote the polymerization speed.

Further, when the polymer of the invention has an acetal bond, it becomes acid decomposable. For example, when a group is bonded to the homoadamantane structure through an acetal bond, if it is used for a photoresist, a bond opposite to the homoadamantane side of an oxygen atom is fractured by an acid, and the group which has been fractured is flown in an alkali, whereby a decrease of roughness or the can be expected.

In the (meth)acrylic polymer of the invention, since an adamantane structure and a lacton structure, which have conventionally been introduced from different monomers, are introduced from the same monomer having them at the same time, it is thought that these structures are dispersed more uniformly in a (meth)acrylic polymer (photoresist resin), leading to reduction of roughness.

A resin composition containing the (meth)acrylic polymer of the invention can be used for various applications, for example, a material for forming a circuit (a photoresist for producing a semiconductor, a print circuit board or the like), an image-forming material (printing plate material, relief image or the like) or the like. In particular, it is preferred that it be used as the resin composition for a photoresist. It is more preferred that the resin composition be used as a resin composition for a positive photoresist.

No specific restrictions are imposed on the positive photoresist composition of the invention as long as they contain the (meth)acrylic polymer or a photoacid generator. However, a positive photoresist composition contains the (meth)acrylic polymer of the invention preferably in an amount of 2 to 50 mass %, more preferably 5 to 15 mass %, relative to 100 mass % of the positive photoresist composition of the invention.

The positive photoresist composition of the invention may contain, in addition to the above-mentioned (meth)acrylic polymer of the invention and the PAG (photoacid generator), quenchers such as an organic amine, an alkali-soluble resin (for example, a NOBOLAC resin, a phenol resin, an imide resin, a carboxyl group-containing resin), a colorant (a dye, for example), an organic solvent (a hydrocarbon, a halogenated hydrocarbon, alcohol, ester, ketone, ether, cellosolve, carbitol, glycol ether ester, and these mixed solvents, or the like).

As the photoacid generator, a generally used compound which efficiently generates an acid by light exposure can be given. Examples thereof include diazonium salts, iodonium salts (for example, diphenyl iodohexafluoro phosphate, or the like), sulfonium salts (for example, triphenylsulfonium hexafluoroantimonate, triphenylsulfonium hexafluorophosphate, triphenylsulfonium methane sulfonate, or the like), sulfonic ester (for example, 1-phenyl-1-(4-methylphenyl)sulfonyloxy-1-benzoylmethane), 1,2,3-trisulfonyloxy methylbenzene, 1,3-dinitro-2-(4-phenylslufonyloxymethyl)benzene, 1-phenyl-1-(4-methylphenylslufonyloxymethyl)-1-hydroxy-1-benzoylmethane), an oxathiazole derivative, an s-triazine derivative, a disulfone derivatives (diphenyl disulfone, or the like), an imide compound, an oxime sulfonate, diazonaphthoquinone, and benzoin tosylate. These photoacid generators can be used singly or in combination of two or more.

The content of the photoacid generator in the positive photoresist composition of the invention can be appropriately selected according to the intensity of an acid generated by light irradiation, the content of a monomer unit based on (meth)acrylic esters of the (meth)acrylic polymer, or the like.

The content of the photoacid generator is preferably 0.1 to 30 parts by mass, more preferably 1 to 25 parts by mass relative to 100 parts by mass of the (meth)acrylic polymer, with 2 to 20 parts by mass being further preferable.

The positive photoresist composition of the invention is prepared by mixing a (meth)acrylic polymer, a photoacid generator, and, according to need, the above-mentioned organic solvent, and removing, if necessary, impurities through a generally-used solid separation means such as a filter.

This positive photoresist composition is applied to a base or a substrate, followed by drying. A coating film (photoresist film) is irradiated with light through a predetermined mask (or, baking is further conducted after light exposure) to form a latent image pattern, followed by development, whereby a minute pattern can be formed with a high degree of accuracy.

The invention also provides a method for forming a resist pattern comprising the steps of forming a photoresist film on a substrate by using the above-mentioned positive photoresist composition, selectively exposing the resist film to light and subjecting the thus selectively irradiated resist film to alkali development to form a photoresist pattern.

As the substrate, silicon wafer, a metal, plastic, glass, ceramics or the like can be given. Formation of a resist film by using a positive photoresist composition can be conducted by means of a generally-used coating means such as a spin coater, a dip coater and a roller coater. The thickness of a photoresist film is preferably 50 nm to 20 μm, more preferably 100 nm to 2 μm.

In selectively exposing a photoresist film to light, light beams with various wavelengths such as UV rays and X rays can be used. For forming a semiconductor photoresist, normally, g rays, i rays, excimer laser (XeCl, KrF, KrCl, ArF, ArCl or the like, for example) and soft X rays are used. Exposure energy is about 0.1 to 1,000 mJ/cm², preferably about 1 to 100 mJ/cm².

The (meth)acrylic polymer contained in the positive photoresist composition of the invention preferably has an acetal structure and has an acid-decomposable function. In this case, an acid is generated from the photoacid generator by the selective light exposure as mentioned above. Due to this acid, of the structural units based on the (meth)acrylic esters in the (meth)acrylic polymer, cyclic parts are removed smoothly, whereby a carboxyl group or a hydroxyl group which contributes to solubilization is generated. Therefore, by conducting development by using an alkaline developer, a prescribed pattern can be formed with a high degree of accuracy.

### EXAMPLES

The invention will be described in more detail with reference to Examples and Comparative Examples which should not be construed as limiting the scope of the invention.

Physical properties were determined as follows:

(1) Nuclear magnetic resonance (NMR): Measured by means of JNM-ECA 500 (manufactured by JEOL Ltd.) by using chloroform-d as the solvent.

(2) Measured by gas chromatography-mass spectrometry (GC-MS): EI (GCMS-QP2010 manufactured by Shimadzu Corporation)

(3) Weight average molecular weight (Mw), degree of dispersion (Mw/Mn): Measured in terms of polystyrene by means of HLC-8220, GPC system (manufactured by Tosoh Corporation, Column=TSGgel G-4000HXL+G-2000HXL)

Using 5-oxo-4-oxa-5-homoadamantane-1-ol was synthesized as follows. Specifically, using 2-adamantanone as a raw material, 4-oxo-1-adamatanol was synthesized by a method described in a literature (J. Org. Chem., 48, 1099-1101 (1983)), followed by a reaction of peroxyformic acid formed of formic acid and a hydrogen peroxide solution.

5-oxo-4-oxa-5-homoadamantane-2-ol was synthesized as follows. Specifically, using 2-adamantanone as a raw material, endo-bicyclo[3.3.1]non-6-en-3-carboxylic acid was synthesized by a method described in a literature (J. Am. Chem. Soc., 108, 15, 4484 (1986)), followed by a reaction of peroxyformic acid formed of formic acid and a hydrogen peroxide solution.

### Example 1

Synthesis of Homoadamantane Derivative: (5-oxo-4-oxa-5-homoadamantane-1-yl)oxymethylchloride

In a 1L-flask, 54.7 g (300 mmol) of 5-oxo-4-oxa-5-homoadamantane-1-ol, 400 mL (5.6 mol) of dimethylsulfoxide (DMSO) and 200 mL (2.1 mol) of acetic anhydride were added, followed by stirring for 3 days. Then, the resultant was subjected to gas chromatography analysis. As a result, it could be confirmed that 5-oxo-4-oxa-5-homoadamantane-1-ol was completely converted to methylthiomethyl ether.

To this reaction mixture liquid, 150 mL of water and 300 mL of diethyl ether were added, and the resultant was shaken and allowed to stand. Thereafter, an aqueous phase and an organic phase were separated. 150 mL of diethyl ether was added again to the aqueous phase, and the resultant was shaken and allowed to stand. Thereafter, an aqueous phase and an organic phase were separated. This procedure was further repeated twice, and the organic phase was dried with magnesium sulfate. The resultant was filtrated and concentrated, and 100 mL of chloroform was added to the resulting yellowish oil, followed by addition of 21.8 mL (300 mmol) of thionyl chloride. After stirring for 1 hour, the solvent and the light gas components were distilled off under reduced pressure, whereby 54.2 g (235 mmol, isolation yield: 78.3%, GC purity: 98.3%) of intended (5-oxo-4-oxa-5-homoadamantane-1-yl)oxymethylchloride represented by the following formula was isolated. Each data of GC-MS, ¹H-NMR and ¹³C-NMR are shown below.

GC-MS: 232 (0.04%), 230 (0.4%), 194 (10.2%), 164 (23.3%), 138 (56.7%), 120 (39.6%), 95 (100%), 79 (58.8%), 67 (22.1%), 55 (18.4%), 41 (29.8%)

¹H-NMR: 1.84˜2.67 (m, 11H), 3.09 (t, J=5.6 Hz, 1H), 4.63 (s, 1H), 5.43 (s, 2H)

¹³C-NMR: 29.69, 30.11, 34.56, 34.63, 38.14, 39.83, 41.37, 68.29, 73.88, 82.07, 177.92

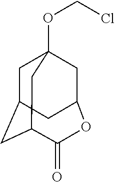 [SMILES: O=C1OC2CC3CC1CC(OCCl)(C3)C2]

### Example 2

Synthesis of a Homoadamantane Derivative: (5-oxo-4-oxa-5-homoadamantane-2-yl)oxymethylchloride

A stirring device was attached to a 1L-separable flask provided with a nozzle for introducing hydrogen chloride gas. To this flask, 54.7 g (300 mmol) of 5-oxo-4-oxa-5-homoadamantane-2-ol, 13.6 g (450 mmol) of paraformaldehyde, 36.2 g (300 mmol) of magnesium sulfate and 650 mL of dried dichloromethane were added, and the resultant was cooled on ice bath to 0° C. and stirred. To this flask, a hydrogen chloride gas generated by mixing 292 g (5.0 mmol) of sodium chloride and 700 mL of concentrated sulfuric acid was blown through the nozzle for 1 hour. Further, after stirring for 3 hours, magnesium sulfate was filtered, and gas chromatography analysis was conducted. As a result, it was confirmed that 5-oxo-4-oxa-5-homoadamantane-2-ol was completely converted into an ether.

Hydrogen chloride and dichlorometane were removed by distillation, whereby 58.1 g (251 mmol, isolation yield: 84.0%, GC purity: 98.9%) of intended (5-oxo-4-oxa-5-homoadamantane-2-yl)oxymethylchloride represented by the following formula was isolated. Each data of GC-MS, ¹H-NMR and ¹³C-NMR are shown below.

GC-MS: 232 (0.01%), 230 (0.5%), 194 (9.9%), 164 (53.7%), 136 (83.0%), 121 (15.8%), 110 (15.9%), 79 (100%), 67 (22.4%), 55 (19.1%), 41 (24.1%)

¹H-NMR: 1.57 (s, 1H), 1.84˜2.25 (m, 9H), 3.22 (s, 1H), 3.92 (s, 1H), 4.11 (s1H), 5.68 (s, 2H)

¹³C-NMR: 24.78, 27.76, 28.53, 29.72, 30.41, 31.67, 38.49, 69.91, 72.30, 82.15, 177.48

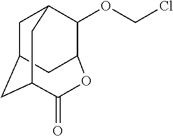 [SMILES: O=C1OC2CC3CC1CC(C3)C2OCCl]

### Example 3

Synthesis of a Homoadamantane Derivative: 2-(5-oxo-4-oxa-5-homoadamantane-1-yl)oxy-2-oxoethylchloride

In a 1L-flask, 36.4 g (200 mmol) of 5-oxo-4-oxa-5-homoadamantane-1-ol was added, and dissolved in 200 mL of tetrahydrofuran. To the resulting mixture, 41.8 mL (300 mmol) of triethylamine was added. While cooling the flask on ice bath, 19.1 mL (240 mmol) of chloroacetyl chloride was slowly added dropwise for about 30 minutes.

Thereafter, stirring was conducted for 3 hours. Then, 100 mL of water was added to terminate the reaction. The resulting reaction mixture was extracted with diethyl ether, washed with water, and dried with anhydrous sodium sulfate. After filtration and concentration, purification was conducted by re-crystallization, whereby 39.8g (154 mmol, isolation yield: 76.9%, GC purity: 97.9%) of intended 2-(5-oxo-4-oxa-5-homoadamantane-1-yl)oxy-2-oxoethylchloride represented by the following formula was isolated. Each data of GC-MS, ¹H-NMR and ¹³C-NMR are shown below.

GC-MS: 261 (0.02%), 259 (0.07%), 214 (0.14%), 164 (14.5%), 138 (35.4%), 120 (26.0%), 105 (15.1%), 95 (64.3%), 93 (43.6%), 92 (100%), 79 (38.1%), 67 (14.5%), 55 (11.6%), 41 (19.8%)

¹H-NMR: 1.80˜2.57 (m, 11H), 3.24 (t, J=5.8 Hz, 1H), 3.99 (t, J=0.8 Hz, 2H), 4.68 (s, 1H)

¹³C-NMR: 29.68, 30.20, 34.55, 34.70, 38.18, 39.70, 41.07, 41.53, 73.67, 80.43, 165.88, 176.75

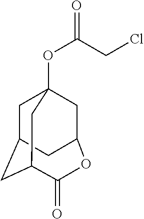 [SMILES: O=C(CCl)OC12CC3CC(C1)OC(=O)C(C3)C2]

### Example 4

Synthesis of a Homoadamantane Derivative: 2-(5-oxo-4-oxa-5-homoadamantane-2-yl)oxy-2-oxoethylchloride

Synthesis was conducted in the same manner as in Example 3, except that 5-oxo-4-oxa-5-homoadamantane-2-ol was used instead of 5-oxo-4-oxa-5-homoadamantane-1-ol, whereby 37.0 g (143 mmol, isolation yield: 71.5%, GC purity: 98.0%) of intended 2-(5-oxo-4-oxa-5-homoadamantane-2-yl)oxy-2-oxoethylchloride represented by the following formula was isolated. Each data of GC-MS, ¹H-NMR and ¹³C-NMR are shown below.

GC-MS: 259 (0.05%), 215 (0.60%), 186 (1.32%), 164 (55.7%), 136 (81.7%), 121 (16.2%), 110 (16.0%), 92 (59.6%), 79 (100%), 67 (22.9%), 55 (18.5%), 41 (24.4%)

¹H-NMR: 1.57 (d, J=13.1 Hz, 1H), 1.86˜2.28 (m, 9H), 3.09 (s, 1H), 4.10 (s, 2H), 4.29 (s, 1H), 5.07 (s, 1 H)

¹³C-NMR: 24.89, 27.76, 28.61, 29.65, 30.34, 31.62, 40.36, 40.65, 72.08, 73.67, 165.71, 176.66

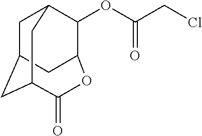 [SMILES: O=C(CCl)OC1C2CC3CC(C2)C(=O)OC1C3]

### Example 5

Synthesis of a Homoadamantane Derivative: 2-(5-oxo-4-oxa-5-homoadamantane-1-yl)oxy-2-oxoethylchloride

In a 1L-flask, 36.4 g (200 mmol) of 5-oxo-4-oxa-5-homoadamantane-1-ol, 1.9 g (10 mmol) of paratoluenesulfonic acid monohydrate and 28.3 g (300 mmol) of chloroacetic acid were added, and dissolved in 500 mL of toluene. The resulting mixture was heated until toluene was boiled, and thereafter, stirring was conducted for 8 hours, and 100 mL of water was added to terminate the reaction. The resulting reaction mixture was washed with water, and then dried with anhydrous sodium sulfate. After filtration and concentration, purification was conducted by re-crystallization, whereby 44.1 g (170 mmol, isolation yield: 85.2%, GC purity: 98.8%) of intended 2-(5-oxo-4-oxa-5-homoadamantane-1-yl)oxy-2-oxoethylchloride represented by the following formula was isolated. Each data of GC-MS, ¹H-NMR and ¹³C-NMR are shown below.

GC-MS: 261 (0.02%), 259 (0.07%), 214 (0.14%), 164 (14.5%), 138 (35.4%), 120 (26.0%), 105 (15.1%), 95 (64.3%), 93 (43.6%), 92 (100%), 79 (38.1%), 67 (14.5%), 55 (11.6%), 41 (19.8%)

¹H-NMR: 1.80˜2.57 (m, 11H), 3.24 (t, J=5.8Hz, 1H), 3.99 (t, J=0.8 Hz, 2H), 4.68 (s, 1H)

¹³C-NMR: 29.68, 30.20, 34.55, 34.70, 38.18, 39.70, 41.07, 41.53, 73.67, 80.43, 165.88, 176.75

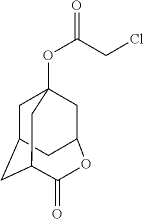 [SMILES: O=C(CCl)OC12CC3CC(C1)OC(=O)C(C3)C2]

### Example 6

Synthesis of a Homoadamantane Derivative: 2-(5-oxo-4-oxa-5-homoadamantane-2-yl)oxy-2-oxoethylchloride

Synthesis was conducted in the same manner as in Example 5, except that 5-oxo-4-oxa-5-homoadamantane-2-ol was used instead of 5-oxo-4-oxa-5-homoadamantane-1-ol was used, whereby 49.1 g (190 mmol, isolation yield: 94.9%, GC purity: 98.0%) of intended 2-(5-oxo-4-oxa-5-homoadamantane-2-yl)oxy-2-oxoethylchloride represented by the following formula was isolated. Each data of GC-MS, ¹H-NMR and ¹³C-NMR are shown below.

GC-MS: 259 (0.05%), 215 (0.60%), 186 (1.32%), 164 (55.7%), 136 (81.7%), 121 (16.2%), 110 (16.0%), 92 (59.6%), 79 (100%), 67 (22.9%), 55 (18.5%), 41 (24.4%)

¹H-NMR: 1.57 (d, J=13.1 Hz, 1H), 1.86˜2.28 (m, 9H), 3.09 (s, 1H), 4.10 (s, 2H), 4.29 (s, 1H), 5.07 (s, 1H)

¹³C-NMR: 24.89, 27.76, 28.61, 29.65, 30.34, 31.62, 40.36, 40.65, 72.08, 73.67, 165.71, 176.66

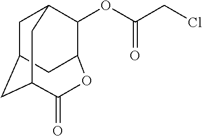 [SMILES: O=C(CCl)OC1C2CC3CC(C2)C(=O)OC1C3]

### Example 7

Synthesis of a Homoadamantane Derivative: 2-(2-(5-oxo-4-oxa-5-homoadamantane-1-yl)oxy-2-oxoethoxy)-2-oxoethanol

In a 500-mL, three-neck flask, 4.6 g (60 mmol) of glycolic acid, 50 mL of DMF, 10.4 g (75 mmol) of potassium carbonate and 3.4 g (20 mmol) of potassium iodide were placed, followed by stirring at room temperature for 30 minutes. To the resulting mixture, 30 mL of a DMF solution of 14.9 g (50 mmol) of 2-(5-oxo-4-oxa-5-homoadamantane-1-yl)oxy-2-oxoethylchloride synthesized in Example 3 was slowly added. The resultant was heated to 45° C., and stirred for 4 hours. After completion of the reaction, 100 mL of toluene was added, followed by filtration. The resulting solution was washed with water, 100 mL of toluene was added, followed by filtration. The resulting solution was washed with water, then with an aqueous 10 wt % sodium thiosulfate, and dried with anhydrous sodium sulfate. After filtration and concentration, re-crystallization was conducted from a toluene-heptane mixed solution, whereby 10.8 g (36.2 mmol, isolation yield: 72.4%, GC purity: 98.7%) of intended 2-(2-(5-oxo-4-oxa-5-homoadamantane-1-yl)oxy-2-oxoethoxy)-2-oxoethanol represented by the following formula was isolated. Each data of GC-MS, ¹H-NMR and ¹³C-NMR are shown below.

GC-MS: 298 (0.02%), 181 (0.09%), 164 (22.7%), 138 (58.4%), 120 (40.6%), 95 (100%), 79 (63.2%), 67 (23.6%), 55 (17.6%), 41 (30.4%)

¹H-NMR: 1.79˜2.55 (m, 11H), 3.36 (t, J=6.0 Hz, 1H), 4.42 (d, J=5.2Hz, 2H), 4.55 (s, 2H), 4.79 (s, 1H)

¹³C-NMR: 29.61, 30.39, 34.67, 34.72, 38.30, 39.85, 41.31, 60.85, 61.13, 74.36, 80.11, 166.23, 171.99, 177.64

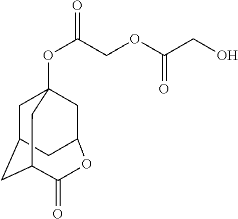 [SMILES: O=C(CO)OCC(=O)OC12CC3CC(C1)OC(=O)C(C3)C2]

### Example 8

Synthesis of a Homoadamantane Derivative: 2-(2-(5-oxo-4-oxa-5-homoadamantane-2-yl)oxy-2-oxoethoxy)-2-oxoethanol

Synthesis was conducted in the same manner as in Example 7, except that 2-(5-oxo-4-oxa-5-homoadamantane-2-yl)oxy-2-oxoethylchloride synthesized in Example 4 was used instead of 2-(5-oxo-4-oxa-5-homoadamantane-1-yl)oxy-2-oxoethylchloride synthesized in Example 3, whereby 11.3 g (37.9 mmol, isolation yield: 75.8%, GC purity: 99.0%) of intended 2-(2-(5-oxo-4-oxa-5-homoadamantane-2-yl)oxy-2-oxoethoxy)-2-oxoethanol represented by the following formula was isolated. Each data of GC-MS, ¹H-NMR and ¹³C-NMR are shown below.

GC-MS: 298 (0.01%), 181 (0.09%), 164 (53.1%), 136 (78.0%), 121 (15.2%), 110 (15.2%), 79 (100%), 67 (22.6%), 55 (18.2%), 41 (23.0%)

¹H-NMR: 1.56 (d, J=12.5 Hz, 1H), 1.80˜2.34 (m, 9H), 3.06 (s, 1H), 4.27 (d, J=5.0 Hz, 2H), 4.34 (s, 1H), 4.94 (s, 1H), 4.99 (s, 2H)

¹³C-NMR: 24.94, 27.78, 28.72, 29.55, 30.19, 31.67, 40.42, 60.78, 61.11, 72.33, 73.81, 165.34, 173.65, 176.38

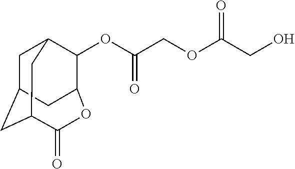 [SMILES: O=C(CO)OCC(=O)OC1C2CC3CC(C2)C(=O)OC1C3]

### Example 9

Synthesis of a Homoadamantane Derivative: 2-(5-oxo-4-oxa-5-homoadamantane-1-yl)oxymethoxy-2-oxoethylchloride

In a 100 mL-flask, 11.5 g (50 mmol) of (5-oxo-4-oxa-5-homoadamantane-1-yl)oxymethylchloride synthesized in Example 1 was put, and dissolved in 50 mL of tetrahydrofuran. Then, 9.1 mL (65 mmol) of triethylamine was added, and stirring was started. To the resultant, 10 mL of a tetrahydrofuran solution of 5.2 g (55 mmol) of chloroacetic acid was slowly added dropwise for about 10 minutes. Subsequently, stirring was conducted for 2 hours, 50 mL of water was added to terminate the reaction. 100 mL of diethyl ether was added to the reaction mixture, washed with water, and then dried with anhydrous sodium sulfate. After filtration and concentration, 13.7 g (47 mmol, isolation yield: 95.0%, GC purity: 95.2%) of intended 2-(5-oxo-4-oxa-5-homoadamantane-1-yl)-oxymethoxy)-2-oxoethylchloride represented by the following formula was isolated. Each data of GC-MS, ¹H-NMR and ¹³C-NMR are shown below.

GC-MS: 288 (0.01%), 260 (1.2%), 258 (3.9%), 164 (24.1%), 194 (21.7%), 138 (54.0%), 120 (40.6%), 95 (100%), 79 (58.5%), 67 (22.5%), 55 (18.6%), 41 (31.0%)

¹H-NMR: 1.88˜2.55 (m, 11H), 3.15 (t, J=5.6 Hz, 1H), 3.91 (s, 2H), 4.45 (s, 1H), 5.63 (s, 2H)

¹³C-NMR: 29.59, 30.29, 34.70, 34.80, 38.06, 39.76, 40.88, 41.10, 74.05, 80.25, 89.25, 167.25, 176.55

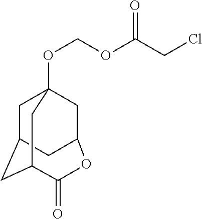 [SMILES: O=C(CCl)OCOC12CC3CC(C1)OC(=O)C(C3)C2]

### Example 10

Synthesis of a Homoadamantane Derivative: 2-(5-oxo-4-oxa-5-homoadamantane-2-yl)oxymethoxy-2-oxoethylchloride

Synthesis was conducted in the same manner as in Example 9, except that (5-oxo-4-oxa-5-homoadamantane-2-yl)oxymethylchloride synthesized in Example 2 was used instead of (5-oxo-4-oxa-5-homoadamantane-1-yl)oxymethylchloride synthesized in Example 1, whereby 12.3 g (43 mmol, isolation yield: 85.0%, GC purity: 95.8%) of intended 2-(5-oxo-4-oxa-5-homoadamantane-2-yl)oxymethoxy-2-oxoethylchloride represented by the following formula was isolated. Each data of GC-MS, ¹H-NMR and ¹³C-NMR are shown below.

GC-MS: 288 (0.01%), 260 (1.3%), 258 (4.1%), 164 (51.9%), 136 (74.8%), 121 (15.5%), 110 (15.5%), 79 (100%), 67 (20.9%), 55 (18.3%), 41 (22.8%)

¹H-NMR: 1.62 (d, J=12.8 Hz, 1H), 1.92˜2.33 (m, 9H), 2.96 (s, 1H), 4.24 (s, 1H), 4.26 (s, 2H), 4.91 (s, 1H), 5.22 (s, 2H)

¹³C-NMR: 24.90, 27.64, 28.73, 29.64, 30.38, 31.63, 40.54, 40.99, 72.17, 73.79, 89.48, 166.30, 177.08

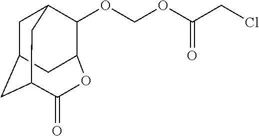 [SMILES: O=C(CCl)OCOC1C2CC3CC(C2)C(=O)OC1C3]

### Example 11

Synthesis of (meth)acrylic Ester: (5-oxo-4-oxa-5-homoadamantane-1-yl)oxymethylmethacrylate

Synthesis was conducted in the same manner as in Example 9, except that 4.7 g (55 mmol) of methacrylic acid was used instead of chloroacetic acid, whereby 13.5 g (48 mmol, isolation yield: 96.3%, GC purity: 97.8%) of intended (5-oxo-4-oxa-5-homoadamantane-1-yl)oxymethylmethacrylate represented by the following formula was isolated. Each data of GC-MS, ¹H-NMR and ¹³C-NMR are shown below.

GC-MS: 250 (30.6%), 194 (59.8%), 164 (23.6%), 138 (53.3%), 120 (41.1%), 95 (100%), 79 (60.9%), 69 (49.6%), 67 (21.8%), 55 (17.4%), 41 (77.1%)

¹H-NMR: 1.73˜2.47 (m, 11H), 2.04 (s, 3H), 3.12 (t, J=5.7 Hz, 1H), 4.53 (s, 1H), 5.29 (s, 2H), 5.67 (t, J=1.5 Hz, 1H), 5.89 (s, 1H)

¹³C-NMR: 18.17, 29.64, 30.13, 34.50, 34.83, 38.36, 39.59, 41.12, 73.80, 80.49, 88.62, 126.52, 136.49, 166.86, 176.74

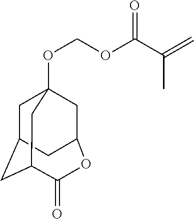 [SMILES: C=C(C)C(=O)OCOC12CC3CC(C1)OC(=O)C(C3)C2]

### Example 12

Synthesis of (meth)acrylic Ester: (5-oxo-4-oxa-5-homoadamantane-2-yl)oxymethylmethacrylate

Synthesis was conducted in the same manner as in Example 11, except that (5-oxo-4-oxa-5-homoadamantane-2-yl)oxymethylchloride synthesized in Example 2 was used instead of (5-oxo-4-oxa-5-homoadamantane-1-yl)oxymethylchloride synthesized in Example 1, whereby 12.9 g (46 mmol, isolation yield: 92.0%, GC purity: 98.2%) of intended (5-oxo-4-oxa-5-homoadamantane-2-yl)oxomethylmethacrylate represented by the following formula was isolated. Each data of GC-MS, ¹H-NMR and ¹³C-NMR are shown below.

GC-MS: 250 (30.8%), 194 (59.0%), 164 (57.2%), 136 (81.2%), 121 (16.6%), 110 (16.1%), 79 (100%), 69 (50.7%), 67 (23.1%), 55 (19.1%), 41 (24.3%)

¹H-NMR: 1.54 (d, J=12.7 Hz, 1H), 1.92˜2.37 (m, 9H), 2.06 (s, 3H), 3.22 (s, 1H), 4.08 (s, 1H), 5.18 (s, 1H), 5.41 (s, 2H), 5.72 (t, J=1.6 Hz, 1H), 5.96 (s, 1H)

¹³C-NMR: 18.15, 24.97, 27.85, 28.51, 29.54, 30.46, 31.74, 40.31, 72.09, 73.83, 88.69, 126.13, 136.70, 166.82, 177.50

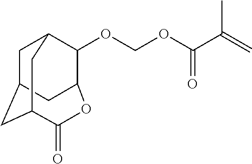 [SMILES: C=C(C)C(=O)OCOC1C2CC3CC(C2)C(=O)OC1C3]

### Example 13

Synthesis of (meth)acrylic Ester: 2-(5-oxo-4-oxa-5-homoadamantane-1-yl)oxy-2-oxoethylmethacrylate

In a 200 mL-three-neck flask, 3.1 mL (36 mmol) of methacrylic acid, 30 mL of DMF, 6.2 g (45 mmol) of potassium carbonate and 2.0 g (12 mmol) of potassium iodide were placed, followed by stirring at room temperature for 30 minutes. To the resultant, 20 mL of a DMF solution of 7.8 g (30 mmol) of 2-(5-oxo-4-oxa-5-homoadamantane-1-yl)oxy-2-oxoethylchloride synthesized in Example 3 was slowly added. The resulting mixture was heated to 45° C., followed by stirring for 4 hours. After the completion of the reaction, 60 mL of toluene was added, and filtered. The resulting solution was washed with water, and then with a 10 wt % aqueous sodium thiosulfate, followed by drying with anhydrous sodium sulfate. After filtration and concentration, purification by silica gel column chromatography, 8.0 g (25.9 mmol, isolation yield: 86.3%, GC purity: 97.5%) of intended 2-(5-oxo-4-oxa-5-homoadamantane-1-yl)oxy-2-oxoethylmethacrylate represented by the following formula was isolated. Each data of GC-MS, ¹H-NMR and ¹³C-NMR are shown below.

GC-MS: 308 (1.0%), 164 (21.3%), 138 (56.6%), 120 (40.9%), 95 (100%), 79 (60.9%), 69 (21.8%), 67 (22.4%), 55 (17.9%), 41 (49.5%)

¹H-NMR: 1.72˜2.55 (m, 11H), 1.98 (s, 3H), 3.18 (t, J=5.6 Hz, 1H), 4.74 (s, 1H), 4.91 (s, 2H), 5.53 (t, J=1.6 Hz, 1H), 6.38 (s, 1H)

¹³C-NMR: 18.16, 29.67, 30.15, 34.65, 34.70, 38.13, 39.71, 41.26, 60.90, 74.21, 80.27, 126.71, 134.76, 166.11, 166.73, 178.11

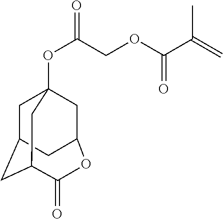 [SMILES: C=C(C)C(=O)OCC(=O)OC12CC3CC(C1)OC(=O)C(C3)C2]

### Example 14

Synthesis of (meth)acrylic Ester: 2-(5-oxo-4-oxa-5-homoadamantane-2-yl)oxy-2-oxoethylmethacrylate

Synthesis was conducted in the same manner as in Example 13, except that 2-(5-oxo-4-oxa-5-homoadamantane-2-yl)oxy-2-oxoethylchloride synthesized in Example 4 was used instead of 2-(5-oxo-4-oxa-5-homoadamantane-1-yl)oxy-2-oxoethylchloride synthesized in Example 3, whereby 7.8 g (25.3 mmol, isolation yield: 84.3%, GC purity: 96.9%) of intended 2-(5-oxo-4-oxa-5-homoadamantane-2-yl)oxy-2-oxoethylmethacrylate represented by the following formula was isolated. Each data of GC-MS, ¹H-NMR and ¹³C-NMR are shown below.

GC-MS: 308 (0.9%), 164 (60.6%), 136 (86.0%), 121 (17.3%), 110 (16.2%), 79 (100%), 69 (20.4%), 67 (23.6%), 55 (19.6%), 41 (45.7%)

¹H-NMR: 1.58 (d, J=13.4 Hz, 1H), 1.76˜2.31 (m, 9H), 2.02 (s, 3H), 3.08 (s, 1H), 4.10 (s, 1H), 4.76 (s, 2H), 5.17 (s, 1H), 5.87 (t, J=1.5 Hz, 1H), 6.35 (s, 1H)

¹³C-NMR: 18.10, 24.85, 27.65, 28.69, 29.57, 30.33, 31.61, 40.50, 61.16, 72.09, 73.63, 126.75, 135.80, 167.25, 167.36, 176.46

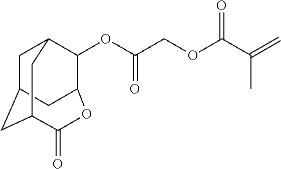 [SMILES: C=C(C)C(=O)OCC(=O)OC1C2CC3CC(C2)C(=O)OC1C3]

### Example 15

Synthesis of (meth)acrylic Ester: 2-(2-(5-oxo-4-oxa-5-homoadamantane-1-yl)oxy-2-oxoethoxy)-2-oxoethylmethacrylate

Synthesis was conducted in the same manner as in Example 13, except that 2-(2-(5-oxo-4-oxa-5-homoadamantane-1-yl)oxy-2-oxoethoxy)-2-oxoethanol synthesized in Example 7 was used instead of 2-(5-oxo-4-oxa-5-homoadamantane-1-yl)oxy-2-oxoethylchloride synthesized in Example 3, whereby 8.4 g (22.9 mmol, isolation yield: 76.3%, GC purity: 97.0%) of intended 2-(2-(5-oxo-4-oxa-5-homoadamantane-1-yl)oxy-2-oxoethoxy)-2-oxoethylmethacrylate represented by the following formula was isolated. Each data of GC-MS, ¹H-NMR and ¹³C-NMR are shown below.

GC-MS: 366 (1.0%), 164 (24.1%), 138 (54.9%), 120 (41.5%), 95 (100%), 79 (58.8%), 69 (32.9%), 67 (22.7%), 55 (18.9%), 41 (57.0%)

¹H-NMR: 1.75˜2.65 (m, 11H), 2.00 (s, 3H), 3.30 (t, J=5.9 Hz, 1H), 4.49 (s, 1H), 4.69 (s, 2H), 4.74 (s, 2H), 5.58 (t, J=1.5 Hz, 1H), 6.11 (s, 1H)

¹³C-NMR: 18.13, 29.62, 30.18, 34.68, 34.74, 38.20, 39.89, 41.09, 60.93, 61.08, 74.27, 80.83, 126.59, 135.40, 166.05, 166.46, 166.74, 177.35

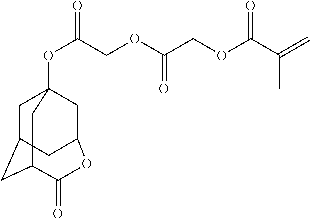 [SMILES: C=C(C)C(=O)OCC(=O)OCC(=O)OC12CC3CC(C1)OC(=O)C(C3)C2]

### Example 16

Synthesis of (meth)acrylic Ester: 2-(2-(5-oxo-4-oxa-5-homoadamantane-2-yl)oxy-2-oxoethoxy)-2-oxoethylmethacrylate

Synthesis was conducted in the same manner as in Example 13, except that 2-(2-(5-oxo-4-oxa-5-homoadamantane-2-yl)oxy-2-oxoethoxy)-2-oxoethanol synthesized in Example 8 was used instead of 2-(5-oxo-4-oxa-5-homoadamantane-1-yl)oxy-2-oxoethylchloride synthesized in Example 3, whereby 8.2 g (22.4 mmol, isolation yield: 74.6%, GC purity: 97.2%) of intended 2-(2-(5-oxo-4-oxa-5-homoadamantane-2-yl)oxy-2-oxoethoxy)-2-oxoethylmethacrylate represented by the following formula was isolated. Each data of GC-MS, ¹H-NMR and ¹³C-NMR are shown below.

GC-MS: 366 (1.0%), 164 (53.4%), 136 (81.0%), 121 (15.4%), 110 (15.8%), 79 (100%), 69 (33.7%), 67 (21.9%), 55 (19.1%) 41 (52.0%)

¹H-NMR: 1.49 (d, J=13.5 Hz, 1H), 1.94 (s, 3H), 1.80˜2.36 (m, 9H), 3.02 (s, 1H), 4.16 (s, 1H), 4.47 (s, 2H), 4.78 (s, 2H), 4.95 (s, 1H), 5.64 (t, J=1.5 Hz, 1H), 6.04 (s, 1H)

¹³C-NMR: 18.07, 24.82, 27.78, 28.52, 29.73, 30.49, 31.72, 40.19, 60.70, 60.74, 71.96, 73.45, 126.78, 135.42, 165.85, 166.10, 166.79, 176.57

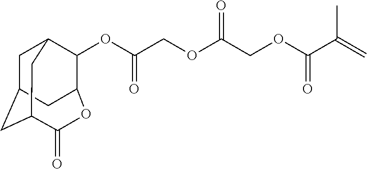 [SMILES: C=C(C)C(=O)OCC(=O)OCC(=O)OC1C2CC3CC(C2)C(=O)OC1C3]

### Example 17

Synthesis of (meth)acrylic Ester: 2-(5-oxo-4-oxa-5-homoadamantane-1-yl)oxymethoxy-2-oxoethylmethacrylate

Synthesis was conducted in the same manner as in Example 13, except that 2-(5-oxo-4-oxa-5-homoadamantane-1-yl)oxymethoxy-2-oxoethylchloride synthesized in Example 9 was used instead of 2-(5-oxo-4-oxa-5-homoadamantane-1-yl)oxy-2-oxoethylchloride synthesized in Example 3, whereby 7.2 g (21.3 mmol, isolation yield: 70.9%, GC purity: 95.3%) of intended 2-(5-oxo-4-oxa-5-homoadamantane-1-yl)oxymethoxy-2-oxoethylmethacrylate represented by the following formula was isolated. Each data of GC-MS, ¹H-NMR and ¹³C-NMR are shown below.

GC-MS: 308 (32.8%), 194 (35.4%), 164 (23.4%), 138 (53.9%), 120 (42.5%), 95 (100%), 79 (60.4%), 69 (50.9%), 67 (21.7%), 55 (17.9%), 41 (70.0%)

¹H-NMR: 1.81˜2.50 (m, 11H), 1.94 (s, 3H), 3.11 (t, J=5.9 Hz, 1H), 4.48 (s, 2H), 4.88 (s, 1H), 5.31 (s, 2H), 5.69 (t, J=1.5 Hz, 1H), 6.48 (s, 1H)

¹³C-NMR: 18.09, 29.64, 30.14, 34.65, 34.73, 38.26, 39.69, 41.46, 60.95, 73.72, 80.24, 88.70, 126.68, 134.80, 166.43, 166.66, 177.11

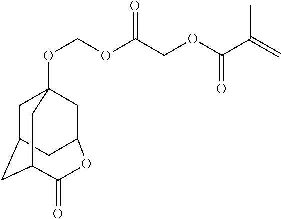 [SMILES: C=C(C)C(=O)OCC(=O)OCOC12CC3CC(C1)OC(=O)C(C3)C2]

### Example 18

Synthesis of (meth)acrylic Ester: 2-(5-oxo-4-oxa-5-homoadamantane-2-yl)oxymethoxy-2-oxoethylmethacryalte

Synthesis was conducted in the same manner as in Example 13, except that 2-(5-oxo-4-oxa-5-homoadamantane-2-yl)oxymethoxy-2-oxoethylchloride synthesized in Example 10 was used instead of 2-(5-oxo-4-oxa-5-homoadamantane-1-yl)oxy-2-oxoethylchloride synthesized in Example 3, whereby 7.6 g (22.5 mmol, isolation yield: 74.9%, GC purity: 95.0%) of intended 2-(5-oxo-4-oxa-5-homoadamantane-2-yl)oxymethoxy-2-oxoethylmethacrylate represented by the following formula was isolated. Each data of GC-MS, ¹H-NMR and ¹³C-NMR are shown below.

GC-MS: 308 (31.5%), 194 (35.0%), 164 (57.5%), 136 (85.3%), 121 (16.5%), 110 (15.5%), 79 (100%), 69 (51.4%), 67 (22.2%), 55 (18.9%), 41 (24.5%)

¹H-NMR: 1.63 (d, J=13.3 Hz, 1H), 1.87˜2.31 (m, 9H), 1.94 (s, 3H), 3.08 (s, 1H), 4.20 (s, 1H), 4.81 (s, 2H), 4.98 (s, 1H), 5.54 (s, 2H), 5.85 (t, J=1.5 Hz, 1H), 6.06 (s, 1H)

¹³C-NMR: 18.19, 24.88, 27.76, 28.66, 29.51, 30.48, 31.76, 40.24, 60.65, 72.29, 73.89, 88.73, 126.84, 135.89, 166.16, 166.28, 175.92

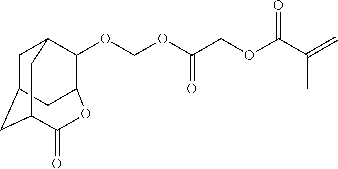 [SMILES: C=C(C)C(=O)OCC(=O)OCOC1C2CC3CC(C2)C(=O)OC1C3]

### Examples 19 to 26

Synthesis of (meth)acrylic Polymer

To methyl isobutyl ketone, 2,2′-azobis(isolactic)dimethyl/monomer A/monomer B/monomer C (compound synthesized in Examples 11 to 18) was incorporated at a mass ratio of 0.1/2.0/1.0/1.0 and the resultant was stirred with heating for 3 hours under reflux. Thereafter, the reaction liquid was poured to a large amount of a mixed solvent of methanol and water to cause precipitation. This operation was repeated three times for purification, whereby copolymers P1 to P8 of each monomer were obtained. The copolymerization composition, the weight average molecular weight (Mw) and the dispersion degree (Mw/Mn) of the copolymers P1 to P8 are shown in Table 1.

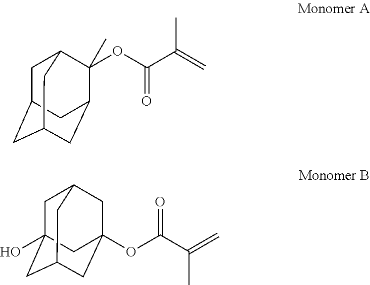

TABLE 1

| | | | | Weight | |
|---|---|---|---|---|---|
| | Examples | | Composition | average | |
| | of | The | of copolymer | molecular | Degree of |
| | monomer | resulting | (A:B:C) | weight | dispersion |
| Examples | C | copolymer | (mol) | (Mw) | (Mw/Mn) |
| 19 | 11 | P1 | 26:35:39 | 6560 | 1.66 |
| 20 | 12 | P2 | 28:31:41 | 6209 | 1.58 |
| 21 | 13 | P3 | 25:34:41 | 6871 | 1.51 |
| 22 | 14 | P4 | 23:34:43 | 7004 | 1.47 |
| 23 | 15 | P5 | 22:31:47 | 6938 | 1.75 |
| 24 | 16 | P6 | 24:31:45 | 7153 | 1.67 |
| 25 | 17 | P7 | 21:33:46 | 7249 | 1.70 |
| 26 | 18 | P8 | 22:29:49 | 6988 | 1.63 |

### Examples 27 to 34

### Preparation of a Positive Photoresist Composition

To 100 parts by mass of each of the copolymers P1 to P8 obtained in Examples 19 to 26, 5 parts by mass of triphenylsulfonium nonafluorobutane sulfonate as the photoacid generator was added. 10 parts by mass of the resulting resin composition was dissolved by using 90 parts by mass of propylene glycol monomethylether acetate, whereby photoresist compositions R1 to R8 were prepared. On a silicon wafer, the thus prepared photoresist compositions R1 to R8 was applied, and baked at 110° C. for 60 seconds to form a photoresist film. The thus obtained wafer was subjected to open exposure at an exposure amount of 100 mJ/cm² with light having a wavelength of 248 nm. Immediately after the exposure, heating was conducted at 110° C. for 60 seconds. Thereafter, development was conducted with an aqueous solution of tetramethyl ammonium hydroxide (2.38 mass %) for 60 seconds. Whether the photoresist film was reduced or not at this time was shown in Table 2. ◯ indicates that the photoresist film was completely removed.

TABLE 2

| | Copolymer | | Reduction in |
|---|---|---|---|
| Examples | used | Resulting photoresist | photoresist film |
| 27 | P1 | R1 | ◯ |
| 28 | P2 | R2 | ◯ |
| 29 | P3 | R3 | ◯ |
| 30 | P4 | R4 | ◯ |
| 31 | P5 | R5 | ◯ |
| 32 | P6 | R6 | ◯ |
| 33 | P7 | R7 | ◯ |
| 34 | P8 | R8 | ◯ |

As mentioned above, it was revealed that the composition containing the (meth)acrylic polymer of the invention functioned as a positive photoresist composition.

### Comparative Example 1

Synthesis of (meth)acrylic Ester: 5-oxo-4-oxa-5-homoadamantane-1-yl methacrylate

Synthesis was conducted in the same manner as in Example 9, except that 9.1 g (50 mmol) of (5-oxo-4-oxa-5-homoadamantane-1-ol) was used instead of (5-oxo-4-oxa-5-homoadamantane-1-yl)oxymethylchloride and 4.7 g (55 mmol) of methacrylic acid was used instead of chloroacetic acid, whereby 11.9 g (48 mmol, isolation yield: 95.1%, GC purity: 98.7%) of intended 5-oxo-4-oxa-5-homoadamantane-1-yl methacrylate represented by the following formula was isolated. Each data of GC-MS, ¹H-NMR and ¹³C-NMR are shown below.

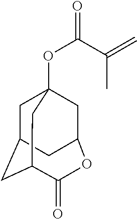 [SMILES: C=C(C)C(=O)OC12CC3CC(C1)OC(=O)C(C3)C2]

### Evaluation Example 1

Synthesis of (meth)acrylic Copolymer

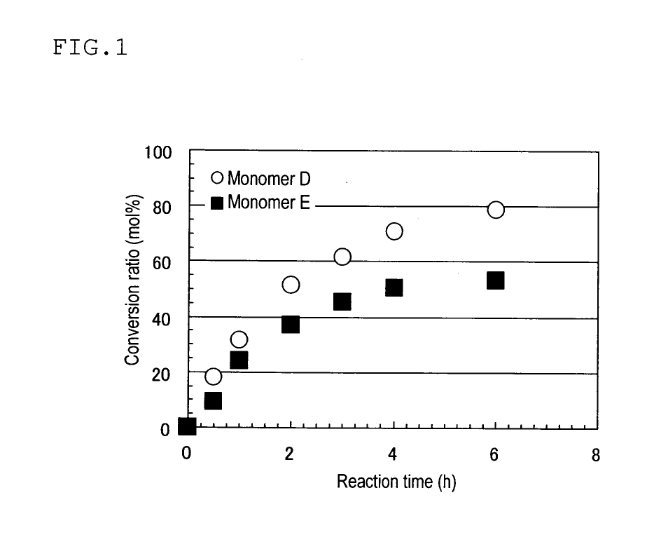

To isobutyl ethyl ketone, 2,2′-azobis(isolactic)dimethyl/monomer D (compound synthesized in Example 13)/monomer E (compound synthesized in Comparative Example 1) were added at a mass ratio of 0.1/1.0/1.0 and the resultant was stirred with heating for 3 hours under reflux. At this time, comparison with the passage of time of the conversion ratio of each monomer was shown in Table 3 and FIG. 1.

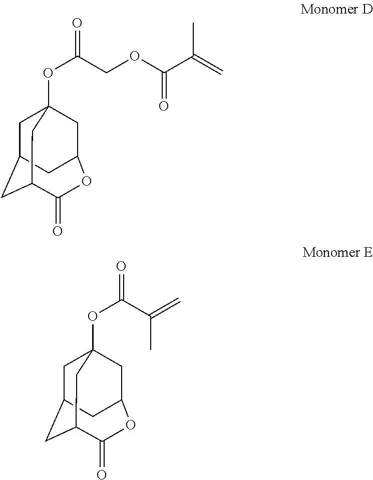 [SMILES: C=C(C)C(=O)OCC(=O)OC12CC3CC(C1)OC(=O)C(C3)C2 ; C=C(C)C(=O)OC12CC3CC(C1)OC(=O)C(C3)C2]

TABLE 3

Conversion ratio (mol %)

| Reaction time (h) | Monomer D | Monomer E |
|---|---|---|
| 0.0 | 0.00 | 0.00 |
| 0.5 | 18.04 | 9.26 |
| 1.0 | 31.48 | 24.25 |
| 2.0 | 51.24 | 36.84 |
| 3.0 | 61.75 | 45.26 |
| 4.0 | 70.86 | 50.57 |
| 6.0 | 78.49 | 53.38 |

As mentioned above, it was confirmed that the (meth)acrylic ester of the present invention also had a high polymerization speed.

### INDUSTRIAL APPLICABILITY

The resin composition comprising the (meth)acrylic polymer of the present invention can be used in a circuit-forming material (a photoresist for producing a semiconductor, a printed circuit board or the like), an image-forming material (a printing board, a relief image or the like) or the like. In particular, the resin composition can be used as a positive photoresist resin composition.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification are incorporated herein by reference in its entirety.

1. A homoadamantane derivative represented by formula (I): 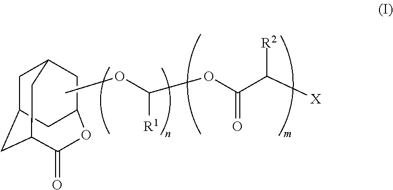
wherein R¹ and R² are each independently a hydrogen atom or a linear, branched or cyclic hydrocarbon group having 1 to 6 carbon atoms, X is a hydroxyl group or a halogen atom, and n and m are independently an integer of 0 to 3, provided that n and m are not both 0.

2. The homoadamantane derivative according to claim 1, which is represented by any of formulas (1) to (3):

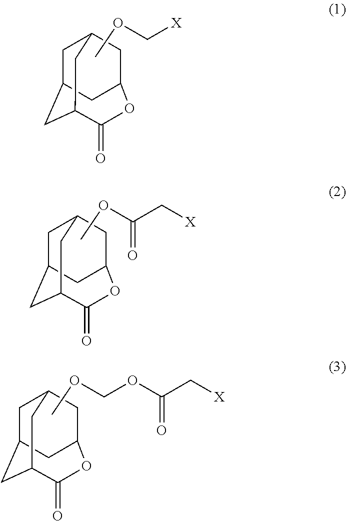

3. The homoadamantane derivative according to claim 2, which is represented by any of formulas (1a) to (3b):

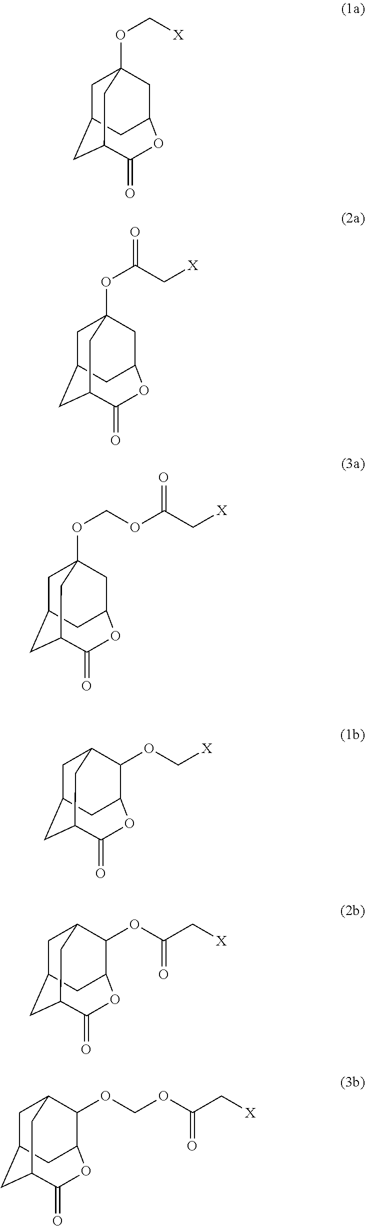

4. A method for producing a homoadmantane derivative according to claim 1, the method comprising at least one process of reacting selected from the group consisting of a, b, c, d and e:
a. reacting homoadamantyl alcohol represented by the following formula, an aldehyde and a halogenated hydrogen gas;
b. reacting homoadamantyl alcohol represented by the following formula with alkylsulfoxide and an acid anhydride to obtain an alkylthioalkyl ether, and reacting this alkylthioalkyl ether with a halogenating agent;
c. reacting homoadmantyl alcohol represented by the following formula with 2-hydroxycarboxylic halide, 2-halogenated carboxylic halide or 2-halogenated carboxylic acid;
d. reacting the halogenated homoadamantane derivative obtained in any of a to c with 2-hydroxycarboxylic acid; and
e. reacting the halogenated homoadamantane derivative obtained in any of a to c with 2-halogenated carboxylic acid

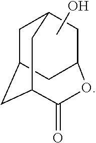

5. A (meth)acrylic ester represented by formula (II): 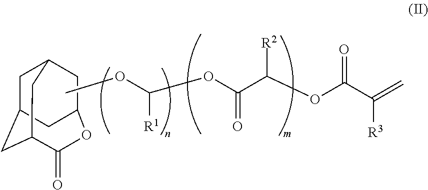
wherein R¹ and R² are each independently a hydrogen atom or a linear, branched or cyclic hydrocarbon group having 1 to 6 carbon atoms and R³ represents a hydrogen atom, a methyl group or a trifluoromethyl group, and n and m are independently an integer of 0 to 3, provided that n and m are not both 0.

6. The (meth)acrylic ester according to claim 5, which is represented by any of formulas (4) to (6):

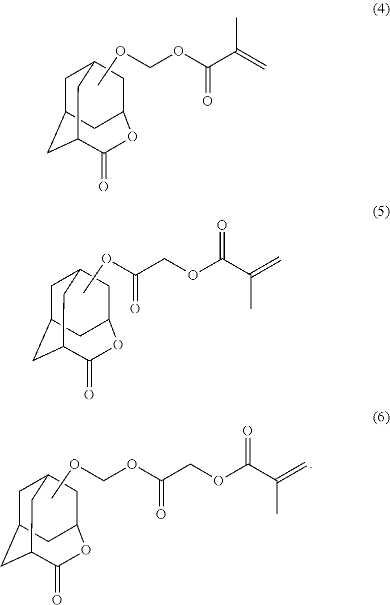

7. The (meth)acrylic ester according to claim 6, which is represented by any of formulas (4a) to (6b):

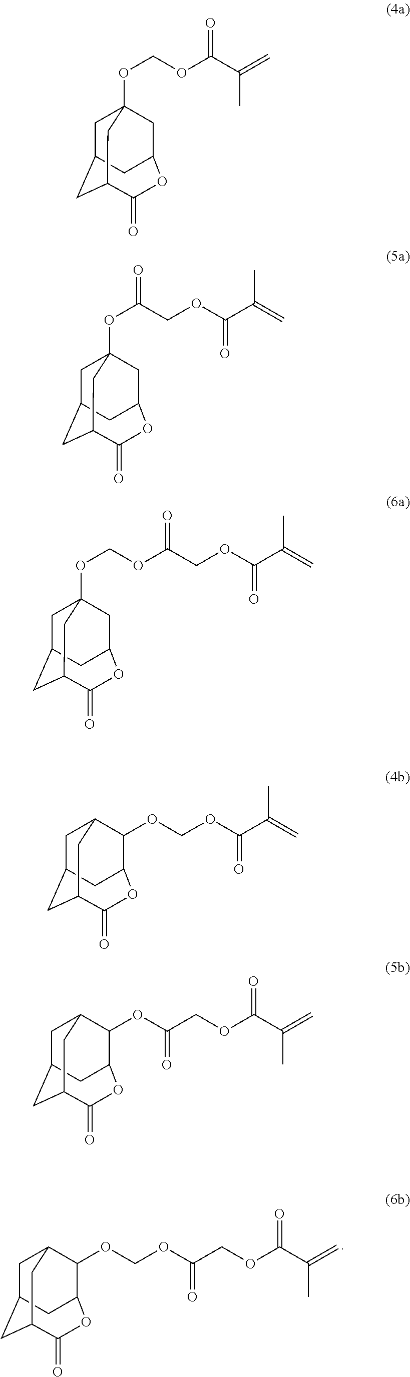

8. A method for producing the (meth)acrylic ester according to claim 5, wherein a homoadamantane derivative represented by formula (I) is reacted with at least one derivative selected from the group consisting of a (meth)acrylic acid derivative, a halide of a (meth)acrylic derivative, an anhydride of a (meth)acrylic derivative and a 2-hydroxyalkyl derivative of a (meth)acrylic acid derivative: 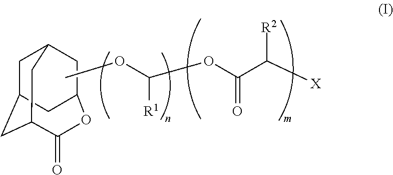
wherein R¹ and R² are each independently a hydrogen atom or a linear, branched or cyclic hydrocarbon group having 1 to 6 carbon atoms, X is a hydroxyl group or a halogen atom, and n and m are independently an integer of 0 to 3, provided that n and m are not both 0.

9. A (meth)acrylic polymer obtained by polymerizing the (meth)acrylic ester according to claim 5.

10. A positive photoresist composition comprising a photoacid generator and the (meth)acrylic polymer according to claim 9.

11. A method for forming a photoresist pattern, the method comprising:
contacting a substrate with the positive photoresist composition according to claim 10, to form a photoresist film;
subjecting the photoresist film to selective exposure; and
subjecting the selectively exposed photoresist film to an alkaline development treatment to form a photoresist pattern.

12. The homoadamantane derivative of claim 1, wherein R¹ and R² are each independently a hydrogen atom or a methyl group.

13. The homoadamantane derivative of claim 1, wherein R¹ and R² are both hydrogen atoms.

14. The homoadamantane derivative of claim 1, wherein X is a hydroxyl group.

15. The homoadamantane derivative of claim 1, wherein X is a halogen atom.

16. The homoadamantane derivative of claim 1, wherein X is a chlorine atom or a bromine atom.

17. The homoadamantane derivative of claim 1, wherein (n, m) are (0, 1), (0, 2), (1, 0), (1, 1), (1, 2), (2, 0), (2, 1) or (2, 2).

18. The homoadamantane derivative of claim 1, wherein (n, m) are (0, 1), (0, 2), (1, 0) or (1, 1).

19. The homoadamantane derivative of claim 3, wherein X is a hydroxyl group.

20. The homoadamantane derivative of claim 3, wherein X is a chlorine atom or a bromine atom.
